SEQUENCE LISTING (83 sequences)

SEQ ID NO: 1 - protein, 5 aa, synthetic construct
  REGION 1..5: note = synthetic construct
RRRRR

SEQ ID NO: 2 - protein, 5 aa, synthetic construct
  REGION 1..5: note = synthetic construct
AAAAA

SEQ ID NO: 3 - protein, 4 aa, synthetic construct
  REGION 1..4: note = synthetic construct
FFFF

SEQ ID NO: 4 - protein, 5 aa, synthetic construct
  REGION 1..5: note = synthetic construct
  MOD_RES 3: note = Phosphocoumaryl amino propionic acid
DEXLI

SEQ ID NO: 5 - protein, 7 aa, synthetic construct
  REGION 1..7: note = synthetic construct
AAAAAAA

SEQ ID NO: 6 - protein, 5 aa, synthetic construct
  REGION 1..5: note = synthetic construct
RARAR

SEQ ID NO: 7 - protein, 5 aa, synthetic construct
  REGION 1..5: note = synthetic construct
DADAD

SEQ ID NO: 8-10 - skipped (3 entries)

SEQ ID NO: 11 - protein, 6 aa, synthetic construct
  REGION 1..6: note = synthetic construct
  MOD_RES 2: note = L-homoproline
  MOD_RES 4: note = L-4-(phosphonodifluoromethyl)phenylalanine
PXGXYR

SEQ ID NO: 12 - skipped

SEQ ID NO: 13 - protein, 6 aa, synthetic construct
  REGION 1..6: note = synthetic construct
  MOD_RES 4: note = L-4-(phosphonodifluoromethyl)phenylalanine
IHIXIR

SEQ ID NO: 14 - protein, 6 aa, synthetic construct
  SITE 2: note = D-alanine
  MOD_RES 4: note = L-4-(phosphonodifluoromethyl)phenylalanine
  MOD_RES 5: note = L-homoproline
AAIXXR

SEQ ID NO: 15 - skipped

SEQ ID NO: 16 - protein, 6 aa, synthetic construct
  MOD_RES 1: note = L-homoproline
  SITE 2: note = D-Asparagine
  MOD_RES 4: note = L-4-(phosphonodifluoromethyl)phenylalanine
XNPXAR

SEQ ID NO: 17 - protein, 6 aa, synthetic construct
  REGION 1..6: note = synthetic construct
  MOD_RES 2: note = L-phenylglycine
  MOD_RES 4: note = L-4-(phosphonodifluoromethyl)phenylalanine
TXAXGR

SEQ ID NO: 18 - protein, 6 aa, synthetic construct
  REGION 1..6: note = synthetic construct
  MOD_RES 4: note = L-4-(phosphonodifluoromethyl)phenylalanine
  SITE 5: note = D-Alanine
AHIXAR

SEQ ID NO: 19 - protein, 6 aa, synthetic construct
  SITE 2: note = D-Asparagine
  MOD_RES 4: note = L-4-(phosphonodifluoromethyl)phenylalanine
  SITE 5: note = D-Proline
GNGXPR

SEQ ID NO: 20 - protein, 6 aa, synthetic construct
  SITE 1: note = D-Phenylalanine
  MOD_RES 3: note = L-homoproline
  MOD_RES 4: note = L-4-(phosphonodifluoromethyl)phenylalanine
FQXXIR

SEQ ID NO: 21 - protein, 6 aa, synthetic construct
  REGION 1..6: note = synthetic construct
  MOD_RES 4: note = L-4-(phosphonodifluoromethyl)phenylalanine
SPGXHR

SEQ ID NO: 22 - protein, 6 aa, synthetic construct
  REGION 1..6: note = synthetic construct
  MOD_RES 1: note = L-homoproline
  MOD_RES 4: note = L-4-(phosphonodifluoromethyl)phenylalanine
XYIXHR

SEQ ID NO: 23 - protein, 6 aa, synthetic construct
  REGION 1..6: note = synthetic construct
  SITE 2: note = D-Valine
  MOD_RES 4: note = L-4-(phosphonodifluoromethyl)phenylalanine
SVPXHR

SEQ ID NO: 24 - protein, 6 aa, synthetic construct
  REGION 1..6: note = synthetic construct
  MOD_RES 4: note = L-4-(phosphonodifluoromethyl)phenylalanine
  SITE 5: note = D-Asparagine
AIPXNR

SEQ ID NO: 25 - protein, 6 aa, synthetic construct
  REGION 1..6: note = synthetic construct
  MOD_RES 1: note = L-4-fluorophenylalanine
  MOD_RES 4: note = L-4-(phosphonodifluoromethyl)phenylalanine
XSIXQF

SEQ ID NO: 26 - protein, 6 aa, synthetic construct
  SITE 2: note = D-Alanine
  MOD_RES 3: note = L-phenylglycine
  MOD_RES 4: note = L-4-(phosphonodifluoromethyl)phenylalanine
  SITE 5: note = D-Phenylalanine
AAXXFR

SEQ ID NO: 27-29 - skipped (3 entries)

SEQ ID NO: 30 - protein, 6 aa, synthetic construct
  SITE 1: note = D-Asparagine
  MOD_RES 3: note = L-4-fluorophenylalanine
  MOD_RES 4: note = L-4-(phosphonodifluoromethyl)phenylalanine
NAXXGR

SEQ ID NO: 31 - protein, 6 aa, synthetic construct
  SITE 1: note = D-Asparagine
  SITE 2: note = D-Threonine
  MOD_RES 4: note = L-4-(phosphonodifluoromethyl)phenylalanine
NTYXAR

SEQ ID NO: 32 - protein, 6 aa, synthetic construct
  SITE 1: note = D-Glutamate
  MOD_RES 3: note = L-phenylglycine
  MOD_RES 4: note = L-4-(phosphonodifluoromethyl)phenylalanine
  SITE 5: note = D-Valine
EAXXVR

SEQ ID NO: 33 - protein, 6 aa, synthetic construct
  SITE 2: note = D-Valine
  MOD_RES 3: note = L-phenylglycine
  MOD_RES 4: note = L-4-(phosphonodifluoromethyl)phenylalanine
IVXXAR

SEQ ID NO: 34 - protein, 6 aa, synthetic construct
  SITE 2: note = D-Threonine
  MOD_RES 3: note = L-phenylglycine
  MOD_RES 4: note = L-4-(phosphonodifluoromethyl)phenylalanine
YTXXAR

SEQ ID NO: 35 - skipped

SEQ ID NO: 36 - protein, 6 aa, synthetic construct
  MOD_RES 1: note = L-homoproline
  SITE 2: note = D-Asparagine
  MOD_RES 4: note = L-4-(phosphonodifluoromethyl)phenylalanine
XNWXHR

SEQ ID NO: 37 - protein, 6 aa, synthetic construct
  MOD_RES 2: note = L-homoproline
  SITE 3: note = D-Valine
  MOD_RES 4: note = L-4-(phosphonodifluoromethyl)phenylalanine
YXVXIR

SEQ ID NO: 38 - protein, 6 aa, synthetic construct
  SITE 1: note = D-Asparagine
  SITE 3: note = D-Alanine
  MOD_RES 4: note = L-4-(phosphonodifluoromethyl)phenylalanine
NSAXGR

SEQ ID NO: 39 - protein, 6 aa, synthetic construct
  SITE 1: note = D-Threonine
  SITE 2: note = D-Asparagine
  SITE 3: note = D-Valine
  MOD_RES 4: note = L-4-(phosphonodifluoromethyl)phenylalanine
  SITE 5: note = D-Alanine
TNVXAR

SEQ ID NO: 40 - protein, 6 aa, synthetic construct
  SITE 1: note = D-Asparagine
  SITE 2: note = D-Threonine
  SITE 3: note = D-Valine
  MOD_RES 4: note = L-4-(phosphonodifluoromethyl)phenylalanine
  SITE 5: note = D-Threonine
NTVXTR

SEQ ID NO: 41 - protein, 6 aa, synthetic construct
  REGION 1..6: note = synthetic construct
  SITE 3: note = D-Threonine
  MOD_RES 4: note = L-4-(phosphonodifluoromethyl)phenylalanine
SITXYR

SEQ ID NO: 42 - protein, 6 aa, synthetic construct
  SITE 1: note = D-Asparagine
  MOD_RES 2: note = L-4-fluorophenylalanine
  SITE 3: note = D-Asparagine
  MOD_RES 4: note = L-4-(phosphonodifluoromethyl)phenylalanine
  SITE 5: note = D-Leucine
NXNXLR

SEQ ID NO: 43 - protein, 6 aa, synthetic construct
  SITE 2..3: note = D-Asparagine
  MOD_RES 4: note = L-4-(phosphonodifluoromethyl)phenylalanine
  SITE 5: note = Nle
YNNXXR

SEQ ID NO: 44 - protein, 6 aa, synthetic construct
  SITE 1: note = D-Asparagine
  SITE 3: note = D-Asparagine
  MOD_RES 4: note = L-4-(phosphonodifluoromethyl)phenylalanine
NYNXGR

SEQ ID NO: 45 - protein, 6 aa, synthetic construct
  REGION 1..6: note = synthetic construct
  SITE 3: note = D-Asparagine
  MOD_RES 4: note = L-4-(phosphonodifluoromethyl)phenylalanine
AWNXAR

SEQ ID NO: 46 - protein, 6 aa, synthetic construct
  SITE 1: note = D-Valine
  SITE 2: note = D-Threonine
  MOD_RES 4: note = L-4-(phosphonodifluoromethyl)phenylalanine
VTHXYR

SEQ ID NO: 47 - skipped

SEQ ID NO: 48 - protein, 6 aa, synthetic construct
  SITE 1: note = D-Asparagine
  MOD_RES 2: note = L-phenylglycine
  MOD_RES 4: note = L-4-(phosphonodifluoromethyl)phenylalanine
NXHXGR

SEQ ID NO: 49 - protein, 6 aa, synthetic construct
  REGION 1..6: note = synthetic construct
  MOD_RES 4: note = L-4-(phosphonodifluoromethyl)phenylalanine
PAHXGR

SEQ ID NO: 50 - protein, 6 aa, synthetic construct
  REGION 1..6: note = synthetic construct
  MOD_RES 4: note = L-4-(phosphonodifluoromethyl)phenylalanine
AYHXIR

SEQ ID NO: 51 - protein, 6 aa, synthetic construct
  SITE 1: note = D-Asparagine
  MOD_RES 2: note = L-homoproline
  SITE 3: note = D-Glutamate
  MOD_RES 4: note = L-4-(phosphonodifluoromethyl)phenylalanine
NXEXYR

SEQ ID NO: 52 - protein, 6 aa, synthetic construct
  SITE 1: note = D-Valine
  MOD_RES 4: note = L-4-(phosphonodifluoromethyl)phenylalanine
  SITE 5: note = D-Threonine
VSSXTR

SEQ ID NO: 53 - protein, 8 aa, synthetic construct
  SITE 1: note = D-Alanine
  MOD_RES 2: note = MeGly
  SITE 3: note = D-Phosphothreonine
  MOD_RES 3: note = Phosphothreonine
  MOD_RES 4: note = L-L-homoprolineecolic acid
  MOD_RES 5: note = L-beta-naphthylalanine
AXXXXYNK

SEQ ID NO: 54 - skipped

SEQ ID NO: 55 - protein, 9 aa, synthetic construct
  SITE 1: note = Trimesic acid moiety attached
  SITE 1: note = D-Alanine
  MOD_RES 2: note = MeGly
  MOD_RES 3: note = Phosphothreonine
  SITE 3: note = D-Phosphothreonine
  MOD_RES 4: note = L-L-homoprolineecolic acid
  MOD_RES 5: note = L-beta-naphthylalanine
  SITE 8: note = D-Alanine
  MOD_RES 9: note = L-2,3-diaminopropionic acid
AXXXXRAAX

SEQ ID NO: 56 - protein, 9 aa, synthetic construct
  SITE 1: note = Trimesic acid moiety attached
  SITE 1: note = D-Alanine
  MOD_RES 2: note = MeGly
  MOD_RES 3: note = D-Threonine
  MOD_RES 4: note = L-L-homoprolineecolic acid
  MOD_RES 5: note = L-beta-naphthylalanine
  SITE 8: note = D-Alanine
  MOD_RES 9: note = L-2,3-diaminopropionic acid
AXTXXRAAX

SEQ ID NO: 57 - protein, 9 aa, synthetic construct
  SITE 1: note = Trimesic acid moiety attached
  SITE 1: note = D-Alanine
  MOD_RES 2: note = MeGly
  SITE 3: note = D-Threonine
  SITE 4: note = D-Alanine
  MOD_RES 5: note = L-beta-naphthylalanine
  SITE 8: note = D-Alanine
  MOD_RES 9: note = L-2,3-diaminopropionic acid
AXTAXRAAX

SEQ ID NO: 58 - protein, 7 aa, synthetic construct
  REGION 1..7: note = synthetic construct
  MOD_RES 2: note = 2-naphthylalanine
FXRRRRQ

SEQ ID NO: 59 - protein, 7 aa, synthetic construct
  SITE 1: note = D-Phenylalanine
  MOD_RES 2: note = 2-naphthylalanine
  SITE 4: note = D-Arginine
  SITE 6: note = D-Arginine
FXRRRRQ

SEQ ID NO: 60 - protein, 8 aa, synthetic construct
  REGION 1..8: note = synthetic construct
  SITE 2: note = D-Phenylalanine
  MOD_RES 3: note = 2-naphthylalanine
  SITE 5: note = D-Arginine
  SITE 7: note = D-Arginine
FFXRRRRQ

SEQ ID NO: 61 - protein, 6 aa, synthetic construct
  REGION 1..6: note = synthetic construct
  MOD_RES 1: note = Phenylalanine with a tert-butanoyl substituent
XRRRRQ

SEQ ID NO: 62 - protein, 6 aa, synthetic construct
  REGION 1..6: note = synthetic construct
  MOD_RES 1: note = 2,3-diaminopropionic acid with a hexanoyl substituent
XRRRRQ

SEQ ID NO: 63 - protein, 6 aa, synthetic construct
  REGION 1..6: note = synthetic construct
  MOD_RES 1: note = 2,3-diaminopropionic acid with an octanoyl substituent
XRRRRQ

SEQ ID NO: 64 - protein, 6 aa, synthetic construct
  REGION 1..6: note = synthetic construct
  MOD_RES 1: note = 2,3-diaminopropionic acid with a decanoyl substituent
XRRRRQ

SEQ ID NO: 65 - protein, 6 aa, synthetic construct
  REGION 1..6: note = synthetic construct
  MOD_RES 1: note = 2,3-diaminopropionic acid with a pyrenoyl substituent
XRRRRQ

SEQ ID NO: 66 - protein, 6 aa, synthetic construct
  REGION 1..6: note = synthetic construct
  MOD_RES 1: note = 2,3-diaminopropionic acid with a 3,3-diphenoyl substituent
XRRRRQ

SEQ ID NO: 67 - protein, 6 aa, synthetic construct
  REGION 1..6: note = synthetic construct
  MOD_RES 1: note = 2,3-diaminopropionic acid with a fluorenylmethyloxycarbonoyl substituent
XRRRRQ

SEQ ID NO: 68 - protein, 6 aa, synthetic construct
  REGION 1..6: note = synthetic construct
  MOD_RES 1: note = 2,3-diaminopropionic acid with a 1-pyrenylbutanoyl substituent
XRRRRQ

SEQ ID NO: 69 - protein, 6 aa, synthetic construct
  MOD_RES 1: note = 2,3-diaminopropionic acid with a decanoyl substituent
  SITE 3: note = D-Arginine
  SITE 5: note = D-Arginine
XRRRRQ

SEQ ID NO: 70 - protein, 6 aa, synthetic construct
  MOD_RES 1: note = 2,3-diaminopropionic acid with a decanoyl substituent
  SITE 2: note = D-Arginine
  SITE 4: note = D-Arginine
XRRRRQ

SEQ ID NO: 71 - protein, 6 aa, synthetic construct
  REGION 1..6: note = synthetic construct
  MOD_RES 1: note = 2,3-diaminopropionic acid with a decanoyl substituent
XARRRQ

SEQ ID NO: 72 - protein, 6 aa, synthetic construct
  REGION 1..6: note = synthetic construct
  MOD_RES 1: note = 2,3-diaminopropionic acid with a decanoyl substituent
XRRRAQ

SEQ ID NO: 73 - protein, 7 aa, synthetic construct
  REGION 1..7: note = synthetic construct
  MOD_RES 1: note = 2,3-diaminopropionic acid with a decanoyl substituent
XRRRRRQ

SEQ ID NO: 74 - protein, 6 aa, synthetic construct
  REGION 1..6: note = synthetic construct
  MOD_RES 1: note = Lysine with a decanoyl substituent
XRRRRQ

SEQ ID NO: 75 - protein, 5 aa, synthetic construct
  REGION 1..5: note = synthetic construct
  MOD_RES 1: note = 2,3-diaminopropionic acid with a decanoyl substituent
XRRRQ

SEQ ID NO: 76 - protein, 6 aa, synthetic construct
  REGION 1..6: note = synthetic construct
  SITE 1: note = Orn with a decanoyl substituent
XRRRRQ

SEQ ID NO: 77 - protein, 6 aa, synthetic construct
  MOD_RES 1: note = Lysine with a decanoyl substituent
  SITE 3: note = D-Arginine
  SITE 5: note = D-Arginine
XRRRRQ

SEQ ID NO: 78 - protein, 6 aa, synthetic construct
  MOD_RES 1: note = Lysine with a decanoyl substituent
  SITE 2: note = D-Arginine
  SITE 4: note = D-Arginine
XRRRRQ

SEQ ID NO: 79 - protein, 6 aa, synthetic construct
  REGION 1..6: note = synthetic construct
  MOD_RES 1: note = Asparagine with a decynoyl substituent
XRRRRQ

SEQ ID NO: 80 - protein, 6 aa, synthetic construct
  MOD_RES 1: note = Asparagine with a decynoyl substituent
  SITE 3: note = D-Arginine
  SITE 5: note = D-Arginine
XRRRRQ

SEQ ID NO: 81 - protein, 6 aa, synthetic construct
  MOD_RES 1: note = Glutamate with a decynoyl substituent
  SITE 3: note = D-Arginine
  SITE 5: note = D-Arginine
XRRRRQ

SEQ ID NO: 82 - protein, 6 aa, synthetic construct
  MOD_RES 1: note = Asparagine with a decynoyl substituent
  SITE 2: note = D-Arginine
  SITE 4: note = D-Arginine
XRRRRQ

SEQ ID NO: 83 - protein, 6 aa, synthetic construct
  MOD_RES 1: note = Glutamate with a decynoyl substituent
  SITE 2: note = D-Arginine
  SITE 4: note = D-Arginine
XRRRRQ

(19) United States
(12) Patent Application Publication
(10) Pub. No.: US 2024/0368227 A1
(43) Pub. Date: Nov. 7, 2024

(54) CYCLIC CELL-PENETRATING PEPTIDES WITH ONE OR MORE HYDROPHOBIC RESIDUES

(71) Applicant: Ohio State Innovation Foundation, Columbus, OH (US)

(72) Inventor: Dehua PEI, Columbus, OH (US)

(21) Appl. No.: 18/644,750

(22) Filed: Apr. 24, 2024

Related U.S. Application Data
(63) Continuation of application No. 17/053,684, filed on Nov. 6, 2020, now Pat. No. 11,987,647.
(60) Provisional application No. 62/669,146, filed on May 9, 2018.

(51) Int. Cl. C07K 7/64

(52) U.S. Cl. CPC C07K 7/64

(57) ABSTRACT

Disclosed are cell penetrating peptides and compositions comprising such peptides that can be used to deliver agents to various cell types.

### CROSS-REFERENCE TO RELATED APPLICATIONS

This application is a continuation of U.S. application Ser. No. 17/053,684, filed Nov. 6, 2020, which is a U.S. national stage application filed under 35 U.S.C. § 371 of International Application No. PCT/US2019/031522, filed May 9, 2019, which claims the benefit of priority to U.S. Provisional Application No. 62/669,146, filed May 9, 2018, the entire contents of which are incorporated herein by reference in its entirety for all purposes.

### ACKNOWLEDGEMENT OF GOVERNMENT SUPPORT

This invention was made with government support under grant no. GM122459 awarded by the National institutes of Health. The government has certain rights in the invention.

### REFERENCE TO AN ELECTRONIC SEQUENCE LISTING

A Sequence Listing conforming to the rules of WIPO Standard ST.26 is hereby incorporated by reference. The Sequence Listing has been filed as an electronic document via EFS-Web in ASCII format encoded as XML. The electronic document, created on Apr. 24, 2024, is entitled “10336-462US2”, and is 146,305 bytes in size.

### BACKGROUND

Traversing the plasma membrane presents a major challenge in drug discovery, especially for biologics such as peptides, proteins and nucleic acids. One potential strategy to subvert the membrane barrier and deliver the biologics into cells is to attach them to “cell-penetrating peptides (CPPs)”. Despite three decades of investigation, the fundamental basis for CPP activity remains elusive. CPPs that enter cells via endocytosis must exit from endocytic vesicles in order to reach the cytosol. Unfortunately, the endosomal membrane has proven to be a significant barrier towards cytoplasmic delivery by these CPPs. What are thus needed are new cell penetrating peptides and compositions comprising such peptides that can be used to deliver agents to various cell types.

The compositions and methods disclosed herein address these and other needs.

### SUMMARY

In various embodiments, the present disclosure provides a cyclic peptide according to one of Formula I-A to I-F:

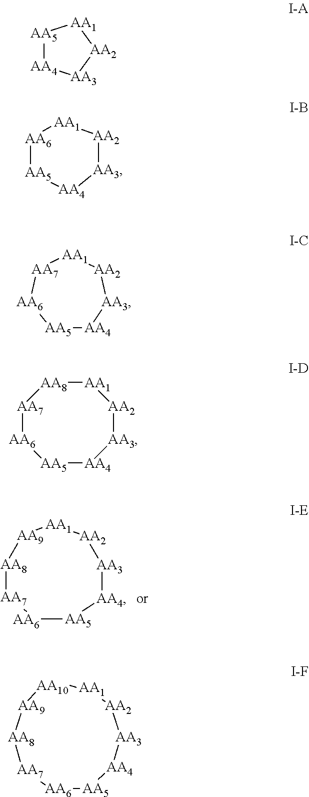

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   at least one of the amino acids is an amino acid having a non-aromatic hydrophobic side chain.

In various embodiments, the present disclosure provides a cyclic peptide according to one of Formula I-A to I-F:

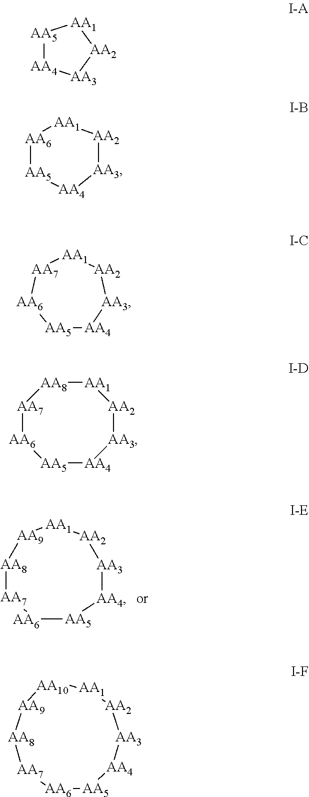

wherein each of AA₁, AA₂, AA₃, AA₄, AA₅, AA₆, AA₇, AA₈, AA₉, and AA₁₀, when
present, are independently selected from an amino acid; and
wherein:
   at least two of the amino acids are arginine;
   at least one of the amino acids is an amino acid having a non-aromatic hydrophobic side chain, provided that the cyclic peptide is not cyclo(GΦRRRRQ), cyclo(GΦRRR), cyclo(GΦRRRQ), or cyclo(GΦRRRR).

In various embodiments, the present disclosure provides a cyclic peptide according to one of Formula IV-A to IV-F:

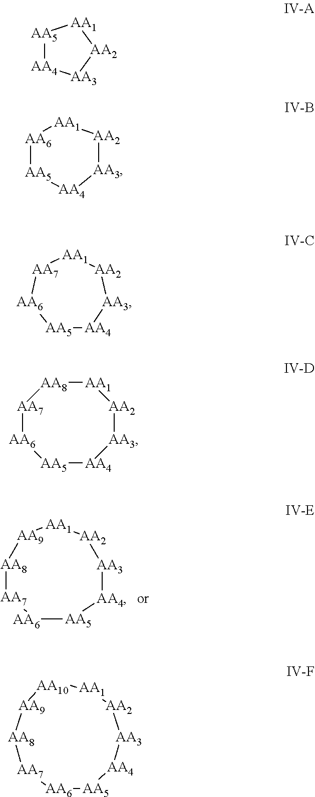

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   one of the amino acids is an amino acid having a hydrophobic side chain; and the remaining amino acids are amino acids having a non-hydrophobic side chain.

In various embodiments, the present disclosure provides a cyclic peptide according to one of Formula IV-A to IV-F:

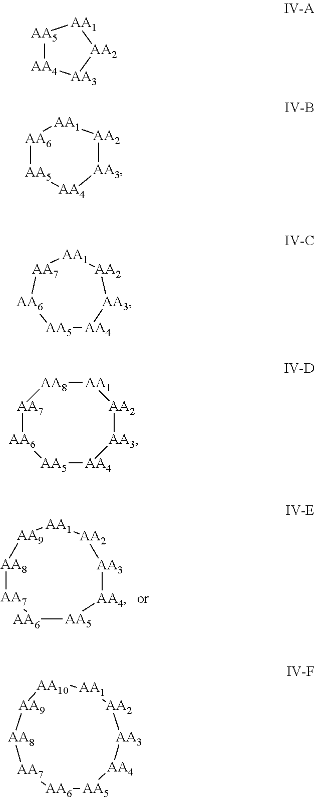

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   one of the amino acids is an amino acid having a hydrophobic side chain; and the remaining amino acids are amino acids having a non-hydrophobic side chain
provided that the cyclic peptide is not cyclo(HΦRRRR), cyclo(YΦRRRR).

In various embodiments, the present disclosure provides a complex comprising a cargo moiety and the cyclic peptide disclosed herein, wherein at least one atom of the cyclic peptide disclosed herein, or at least one lone pair of the cyclic peptide disclosed herein, forms a bond to the cargo moiety.

### BRIEF DESCRIPTION OF THE FIGURES

FIG. 1 shows the structures of cyclic CPP1-1 to CPP1-9.

FIG. 2 is a graph showing the cytosolic entry efficiency of CPP1 and CPP1-1 to CPP1-9 as determined by flow cytometry analysis.

FIG. 3A compares the cytosolic entry efficiencies of CPP1-4, CPP1-15, CPP1-17, CPP1-20, CPP9, and CPP12 into HeLa cells in the presence of 10% FBS; and FIG. 3B compares the cytosolic entry efficiencies of CPP1-4, CPP1-15, CPP1-17, CPP1-20, CPP9, and CPP12 into HeLa cells in the presence of 1% FBS.

FIG. 4 illustrates that effect of CPP1-15 on the viability of HeLa cells.

FIGS. 5A-5B shows live-cell confocal images of HeLa cells after 2-h treatment of 5 μM FITC-CPP1-15 (FIG. 5A) or FITC-CPP1-20 (FIG. 5B) in the presence of 1% FBS. I, FITC fluorescence; II, DIC.

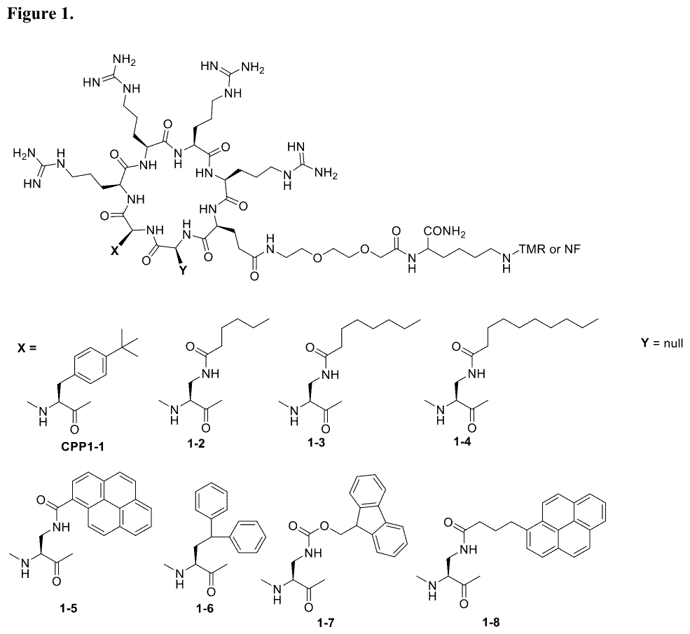

### DETAILED DESCRIPTION

### Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It will also be understood that when a range is provided, said range encompasses each and every value and subrange within the range.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

The terms “peptide,” “protein,” and “polypeptide” are used interchangeably to refer to a natural or synthetic molecule comprising two or more amino acids linked by the carboxyl group of one amino acid to the alpha amino group of another.

As used herein, the term “adjacent” refers to two contiguous amino acids, which are connected by a covalent bond. For example, in the context of a representative cyclic peptide such as

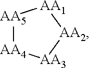

AA₁/AA₂, AA₂/AA₃, AA₃/AA₄, and AA₅/AA₁ exemplify pairs of adjacent amino acids. The term “adjacent” can also be applied to amino acids in a linear sequence, i.e, an acyclic peptide.

A residue of a chemical species, as used herein, refers to a derivative of a moiety that is present in a particular product. To form the product, at least one atom of the moiety is replaced by a bond to a second moiety, such that the product contains a derivative of a moiety. For example, in some embodiments, an aromatic residue in a product may refer to one or more —(C₆H₅)ₙ units present in a cyclic peptide described herein. Similarly, an amino acid residue in a product may refer to cyclic peptide described herein having an amino acid incorporated therein through formation of one or more peptide bonds, and such residues may be referred to interchangeably herein as an amino acid or an amino acid residue.

As used herein, the term “chirality” refers to the “D” and “L” isomers of amino acids or amino acid residues.

As used herein, the term “non-aromatic hydrophobic” refers to a moiety that is not soluble in water and which does not comprise an aromatic ring. Generally, neutral moieties and/or non-polar moieties, or moieties that are predominately neutral and/or non-polar are hydrophobic. Hydrophobic can be measured by one of the methods disclosed herein below. Non-aromatic hydrophobic residues include saturated and unsaturated carbocyclyl and heterocyclyl groups which are not aromatic, as well as alkyl, alkenyl, and alkynyl. In some embodiments, the term “non-aromatic hydrophobic” can include groups in which a hydrophobic residue to attached to rest of the molecule through a bonding group which otherwise could be considered to be polar, such as acyl and alkylcarboxamidyl groups as defined below.

As used herein “aromatic” refers to an unsaturated cyclic molecule having 4n+2π electrons, wherein n is any integer. The term “non-aromatic” refers to any unsaturated cyclic molecule which does not fall within the definition of aromatic.

The term “acyl” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to forty carbon atoms, and which is attached to the rest of the molecule by a single bond. Alkyls comprising any number of carbon atoms from 1 to 20 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Alkenyl group comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl(vinyl), 1-propenyl, 2-propenyl(allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group —O—C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R_{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O)OR, where R is alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂—R, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetereoaryl, as defined herein. Unless stated otherwise specifically in the specification, arylthio can be optionally substituted.

“Alkynyl” or “alkynyl group” refers to a straight or branched hydrocarbon chain radical having from two to forty carbon atoms, and having one or more carbon-carbon triple bonds. Each alkynyl group is attached to the rest of the molecule by a single bond. Alkynyl group comprising any number of carbon atoms from 2 to 40 are included. An alkynyl group comprising up to 40 carbon atoms is a C₂-C₄₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain, having from two to forty carbon atoms, and having one or more carbon-carbon triple bonds. Non-limiting examples of C₂-C₄₀ alkynylene include ethynylene, propargylene and the like. Unless stated otherwise specifically in the specification, an alkynylene chain can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include cycloalkyl, cycloalkenyl, and cycloalkynyl as defined herein. In some embodiments, the carbocyclyl is monovalent and is attached to the rest of molecule through a single bond. In some embodiments, the carbocyclyl is divalent and is independently attached to two moieties through single bonds. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl,” “heterocyclic ring” or “heterocycle” refers to a stable 3- to 20-membered non-aromatic ring radical, which consists of two to fourteen carbon atoms and from one to eight heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl is monovalent and is attached to the rest of molecule through a single bond. In some embodiments, the heterocyclyl is divalent and is independently attached to two moieties through single bonds. Unless stated otherwise specifically in the specification, a heterocyclyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups —OAr, where Ar is an aryl or heteroaryl group as defined herein. Unless otherwise stated specifically in the specification, the aryloxy group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl(benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” refers to a radical of the formula —R_{b}—R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

### Cell Penetrating Peptides

Disclosed herein are cyclic peptides having activity as cell penetrating peptides (cCPPs). In some embodiments, the cCPPs include any combination of at least two arginines and either at least one amino acid having a non-aromatic hydrophobic side chain or only one amino acid having a hydrophobic side chain, with a total number of amino acids in the cCPP in the range of from 4 to about 20 amino acids. In some embodiments, the cCPPs disclosed herein comprise about 4 to about to about 13 amino acids, e.g., about 5, about 6, about 7, about 8, about 9, about 10, or about 11 amino acids, or about 12 amino acids, inclusive of all ranges and subranges therebetween. In particular embodiments, the cCPPs disclosed herein comprise from about 5 to about 12 amino acids, from about 6 to about 10 amino acids, or from about 6 to about 8 amino acids.

Prior to the present invention, the presence of two hydrophobic aromatic side chains was believed to be critical to efficient cellular uptake. Suprisingly, the inventors discovered that a one of the hydrophobic aromatic side chains can be replaced with a non-aromatic hydrophobic side chain and produce cCPPs with high cytosolic delivery efficiency. In addition, the inventors surprisingly discovered that a cCPP can be synthesized with only one hydrophobic side chain (which may be aromatic or non-aromatic) and still achieve high cytosolic delivery efficency.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative, or combinations thereof. Analogs of amino acids encompass that have a structural similar but not identical to an amino acid, e.g., due to a modification to the side chain or backbone on said amino acid. Such modifications may increase the hydrophobicity of the side chain, including elongation of the side chain by one or more hydrocarbons, or increasing the the solvent accessible surface area (SASA as described herein) of an amino acid having an aromatic ring on its side chain, e.g., by conjugating a second aromatic ring or increasing the size of the aromatic ring. Derivatives of amino acids encompass natural and non-natural amino acids that have been modified (e.g., by susbstitution) to include a hydrophobic group as described herein. For example, a derivative of lysine includes lysine whose side chain has been substituted with alkylcarboxamidyl. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allosoleucine | AIle | aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Gistidine | His (H) | his (h) |
| Homoproline (aka pipecolic acid) | Pip (Θ) | Pip (θ) |
| Isoleucine | Ile (I) | ile (i) |
| Leucine | Leu (L) | leu (l) |
| Lysine | Lys (K) | lys (k) |
| Methionine | Met (M) | met (m) |
| Napthylalanine | Nal (Φ) | nal (ϕ) |
| Norleucine | Nle (Ω) | nle |
| Phenylalanine | Phe (F) | phe (F) |
| Phenylglycine | Phg (Ψ) | phg |
| 4-(phosphonodifluoromethyl) | F₂Pmp (Λ) | f₂pmp |
| phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | bta |

*single letter abbreviations: when shown in capital letters herein it indicates the L-amino acid form, when shown in lower case herein it indicates the D-amino acid form.

In some embodiments, the present disclosure provides for cyclic peptide having a structure according to Formula I:

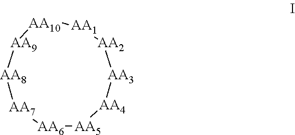

wherein:
   each of AA₁, AA₂, AA₃, AA₄, AA₅, and AA₆ are independently selected from an amino acid; and
   each of AA₇, AA₈, AA₉, and AA₁₀, are independently absent or selected from an amino acid; and
wherein:
   at least two of AA₁, AA₂, AA₃, AA₄, AA₅, AA₆, AA₇, AA₈, AA₉, or AA₁₀ are arginine; and
   either:
      at least one of AA₁, AA₂, AA₃, AA₄, AA₅, AA₆, AA₇, AA₈, AA₉, or AA₁₀ is an amino acid having a non-aromatic hydrophobic side chain, or
      one of AA₁, AA₂, AA₃, AA₄, AA₅, AA₆, AA₇, AA₈, AA₉, or AA₁₀ is an amino acid having a hydrophobic side chain (which may be aromatic or non-aromatic), and the remaining amino acids are amino acids having a non-hydrophobic side chain.

The amino acids, and arrangement thereof, of Formula I are described in detail below in Formulae I-A to I-F and Formulae IV-A to IV-F.

cCPPs with at Least One Amino Acid Having a Hydrophobic Non-Aromatic Side Chain

In some embodiments, the cCPPs disclosed herein (i.e., the cyclic peptides of Formula I) have a structure according to any of Formula I-A to I-F:

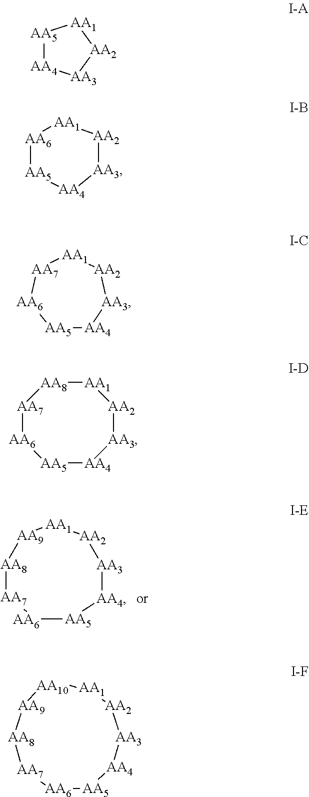

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   at least one of the amino acids is an amino acid having a non-aromatic hydrophobic side chain.

In some embodiments, the cCPPs disclosed herein (i.e., the cyclic peptides of Formula I) have a structure according to any of Formula I-A to I-F:

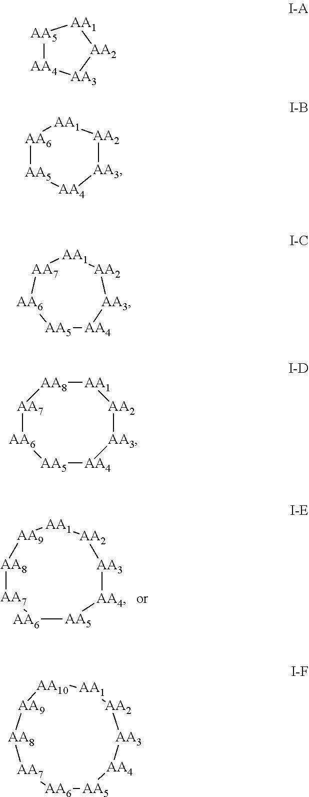

wherein each of AA₁, AA₂, AA₃, AA₄, AA₅, AA₆, AA₇, AA₈, AA₉, and AA₁₀, when present, are independently selected from an amino acid; and
wherein:
at least two of the amino acids are arginine;
at least one of the amino acids is an amino acid having a non-aromatic hydrophobic side chain, provided that the cyclic peptide is not cyclo(GDRRRRQ), cyclo(GDRRR), cyclo(GDRRRQ), or cyclo(GDRRRR).

When the cyclic peptide has a structure according to Formula I-A, at least two of AA₁, AA₂, AA₃, AA₄, and AA₅ are arginine. When the cyclic peptide has a structure according to Formula I-B, at least two of AA₁, AA₂, AA₃, AA₄, AA₅, and AA₆ are arginine. When the cyclic peptide has a structure according to Formula I-C, at least two of AA₁, AA₂, AA₃, AA₄, AA₅, AA₆, and AA₇ are arginine. When the cyclic peptide has a structure according to Formula I-D, at least two of AA₁, AA₂, AA₃, AA₄, AA₅, AA₆, AA₇, and AA₈ are arginine. When the cyclic peptide has a structure according to Formula I-E, at least two of AA₁, AA₂, AA₃, AA₄, AA₅, AA₆, AA₇, AA₈, and AA₉ are arginine. When the cyclic peptide has a structure according to Formula I-F, at least two of AA₁, AA₂, AA₃, AA₄, AA₅, AA₆, AA₇, AA₈, AA₉, and AA₁₀ are arginine. Therefore, the cyclic peptide sequences according to Formula I, I-A, I-B, I-C, I-D, I-E, and I-F include at least two arginines.

cCPPs with at Only One Amino Acid Having a Hydrophobic Side Chain

In various embodiments, the cCPPs disclosed herein (i.e. the cyclic peptides of Formula I) have structure according to one of Formula IV-A to IV-F:

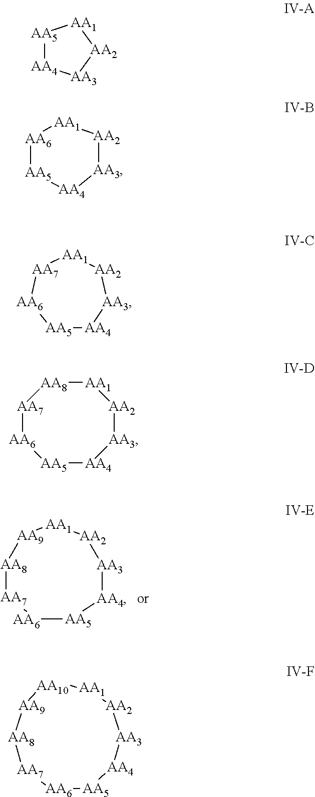

wherein each of AA₁, AA₂, AA₃, AA₄, AA₅, AA₆, AA₇, AA₈, AA₉, and AA₁₀, when present, are independently selected from an amino acid; and
wherein:
   at least two of the amino acids are arginine;
   one of the amino acids is an amino acid having a hydrophobic side chain (which may be aromatic or non-aromatic); and
   the remaining amino acids are amino acids having a non-hydrophobic side chain.

In various embodiments, the present disclosure provides a cyclic peptide according to one of Formula IV-A to IV-F:

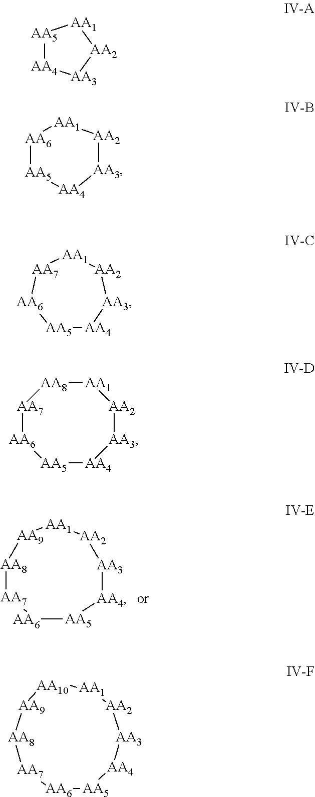

wherein each of AA₁, AA₂, AA₃, AA₄, AA₅, AA₆, AA₇, AA₈, AA₉, and AA₁₀, when present, are independently selected from an amino acid; and wherein:
   at least two of the amino acids are arginine;
   one of the amino acids is an amino acid having a hydrophobic side chain; and the remaining amino acids are amino acids having a non-hydrophobic side chain
provided that the cyclic peptide is not cyclo(HΦRRRR) or cyclo(YΦRRRR).

In some embodiments, the cyclic peptide is not cyclo(HΦRRRRQ) or cyclo(YΦRRRRQ).

When the cyclic peptide has a structure according to Formula IV-A, at least two of AA₁, AA₂, AA₃, AA₄, and AA₅ are arginine. When the cyclic peptide has a structure according to Formula IV-B, at least two of AA₁, AA₂, AA₃, AA₄, AA₅, and AA₆ are arginine. When the cyclic peptide has a structure according to Formula IV-C, at least two of AA₁, AA₂, AA₃, AA₄, AA₅, AA₆, and AA₇ are arginine. When the cyclic peptide has a structure according to Formula IV-D, at least two of AA₁, AA₂, AA₃, AA₄, AA₅, AA₆, AA₇, and AA₈ are arginine. When the cyclic peptide has a structure according to Formula IV-E, at least two of AA₁, AA₂, AA₃, AA₄, AA₅, AA₆, AA₇, AA₈, and AA₉ are arginine. When the cyclic peptide has a structure according to Formula IV-F, at least two of AA₁, AA₂, AA₃, AA₄, AA₅, AA₆, AA₇, AA₈, AA₉, and AA₁₀ are arginine. Therefore, the cyclic peptide sequences according to Formula I, IV-A, IV-B, IV-C, IV-D, IV-E, and IV-F include at least two arginines.

### Hydrophobic Amino Acids

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, 3-(3-benzothienyl)-alanine, or norleucine, each of which is optionally substituted with one or more substituents.

In some embodiments, the amino acid having a hydrophobic side chain is independently an amino acid having a hydrophobic non-aromatic side chain. In some embodiments, an amino acid having a hydrophobic non-aromatic side chain is glycine, alanine, valine, leucine, isoleucine, methionine, or proline. In other embodiments, the amino acid having a hydrophobic non-aromatic side chain has a side chain comprising a C₅-C₄₀ alkyl, alkenyl, alkynyl, acyl, alkylcarboxamidyl, alkoxycarbonyl, carbocyclyl, or heterocyclyl.

In some embodiments, the amino acid having a hydrophobic side chain is independently an amino acid having a hydrophobic aromatic side chain. In some embodiments, an amino acid having a hydrophobic aromatic side chain is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

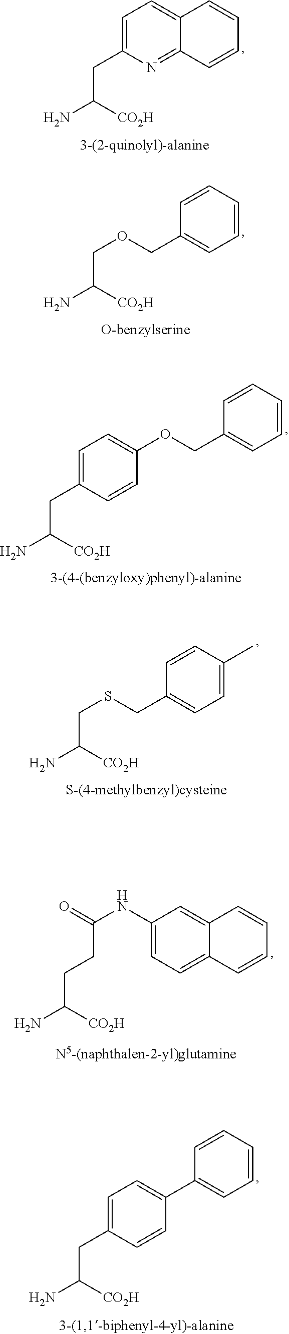 [SMILES: NC(Cc1ccc2ccccc2n1)C(=O)O ; NC(COCc1ccccc1)C(=O)O ; NC(Cc1ccc(OCc2ccccc2)cc1)C(=O)O ; Cc1ccc(CSCC(N)C(=O)O)cc1 ; NC(CCC(=O)Nc1ccc2ccccc2c1)C(=O)O ; NC(Cc1ccc(-c2ccccc2)cc1)C(=O)O]

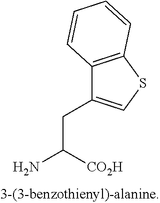 [SMILES: NC(Cc1csc2ccccc12)C(=O)O]

Those skilled in the art will appreciate that the N- and/or C-termini of the above non-natural aromatic hydrophobic amino acids, upon incorporation into the peptides disclosed herein, form amide bonds.

The optional substituent can be any atom or group which does not significantly reduce the cytosolic delivery efficiency of the cCPP, e.g., a substituent that does not reduce the relative cytosolic delivery efficiency to less than that of c (FΦRRRRQ). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein below) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

Amino acids having higher hydrophobicity values can be selected to improve cytosolic delivery efficiency of a cCPP relative to amino acids having a lower hydrophobicity value. In some embodiments, each hydrophobic amino acid independently has a hydrophobicity value which is greater than that of glycine. In other embodiments, each hydrophobic amino acid independently is a hydrophobic amino acid having a hydrophobicity value which is greater than that of alanine. In still other embodiments, each hydrophobic amino acid independently has a hydrophobicity value which is greater or equal to that of phenylalanine. Hydrophobicity may be measured using hydrophobicity scales known in the art. Table 2 below lists hydrophobicity values for various amino acids as reported by Eisenberg and Weiss (Proc. Natl. Acad. Sci. U.S.A. 1984; 81(1):140-144), Engleman, et al. (Ann. Rev, of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference in its entirety. In particular embodiments, hydrophobicity is measured using the hydrophobicity scale reported in Engleman, et al.

TABLE 2

| Amino | | Eisenberg | Engleman | Kyrie and | Hoop and | |
|---|---|---|---|---|---|---|
| Acid | Group | and Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

In some embodiments, an arginine is adjacent to a hydrophobic amino acid. In some embodiments, the arginine has the same chirality as the hydrophobic amino acid. In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two adjacent arginines. In further embodiments, one hydrophobic amino acid is adjacent to one of the arginines. In still other embodiments, the CPPs described herein comprise at least three adjacent hydrophobic amino acids and at least three adjacent arginines. In further embodiments, one hydrophobic amino acid is adjacent to one of the arginines. These various combinations of amino acids can have any arrangement of D and L amino acids, e.g., any of the sequences described in the preceding paragraph.

In some embodiments, any four adjacent amino acids in the cCPPs described herein (e.g., cCPPs of Formulae IA-IF and IVA-IVF) can have one of the following sequences: AA_{H1} -R—R—R or R—R—R-AA_{H1}, wherein each AA_{H1} is independently an amino acid having a hydrophobic side chain.

Accordingly, in some embodiments, the cCPPs herein (e.g., cCPPs of Formulae IA-IF and IVA-IVF) can have a structure according any of Formula III-A to III-L:

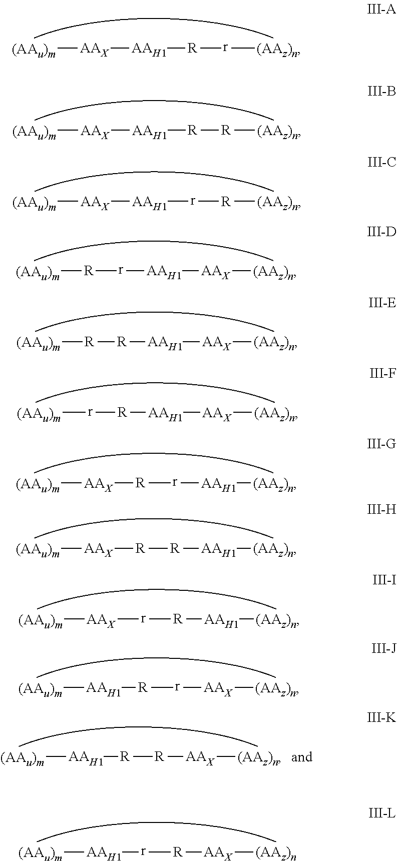

wherein:
AA_{H1} is an amino acid having a non-aromatic hydrophobic side chain;
at each instance AAᵤ, AAₓ, and AAz are independently any amino acid;
wherein:
each of m and n are independently a number from 0 to 6, provided that at least one of m or n is not 0 and the total number of amino acids is from 5 to 10.

In embodiments in which the cCCP has only one hydrophobic side chain (cCPPs according to Formulae IVA-IVF), none of AAᵤ, AAₓ, and AAz are amino acids having a hydrophobic side chain.

In certain embodiments, the cCPPs herein (e.g., cCPPs of Formulae IA-IF and IVA-IVF) is:

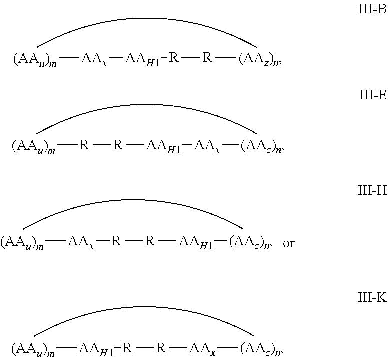

wherein AAH1, AAu, AAx, AAz, m, and n are as defined above.

In some embodiments, the total number of amino acids (including r, R, AA_{H1}, AAᵤ, AAₓ, and AA_{z}), in the cCPPs of Formula III-A to III-L is in the range of 5 to 10. In some embodiments, the total number of amino acids is 5. In some embodiments, the total number of amino acids is 6. In some embodiments, the total number of amino acids is 7. In some embodiments, the total number of amino acids is 8. In some embodiments, the total number of amino acids is 9. In some embodiments, the total number of amino acids is 10.

In some embodiments, the sum of m and n is from 2 to 6. In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, or norleucine, each of which is optionally substituted with one or more substituents.

In some embodiments, the amino acid having a hydrophobic side chain is independently an amino acid having a hydrophobic non-aromatic side chain. In some embodiments, an amino acid having a hydrophobic non-aromatic side chain is glycine, alanine, valine, leucine, isoleucine, methionine, or proline. In other embodiments, the amino acid having a hydrophobic non-aromatic side chain is a non-natural amino acid and has a side chain comprising a C₅-C₄₀ alkyl, alkenyl, alkynyl, acyl, alkylcarboxamidyl, alkoxycarbonyl, carbocyclyl, or heterocyclyl.

In some embodiments, the amino acid having a hydrophobic side chain is independently an amino acid having a hydrophobic aromatic side chain. In some embodiments, an amino acid having a hydrophobic aromatic side chain is naphthylalanine, phenylglycine, homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

In some embodiments, AAH₁ is a hydrophobic amino acid having a hydrophobicity value which is greater than that of glycine. In other embodiments, AAH₁ is a hydrophobic amino acid having a hydrophobicity value which is greater than that of alanine. In still other embodiments, each AAH₁ is a hydrophobic amino acid having a hydrophobicity value which is greater than that of phenylalanine, e.g., as measured using the hydrophobicity scales described above, including Eisenberg and Weiss (Proc. Natl. Acad. Sci. U.S.A. 1984; 81(1):140-144), Engleman, et al. (Ann. Rev. of Biophys. Biophys. Chem. 1986; 1986(15):321-53), Kyte and Doolittle (J. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), (see Table 1 above). In particular embodiments, hydrophobicity is measured using the hydrophobicity scale reported in Engleman, et al.

The presence of a hydrophobic amino acid on the N- or C-terminus of a D-Arg or L-Arg, or a combination thereof, has also been found to improve the cytosolic uptake of the CPP (and the attached cargo). For example, in some embodiments, the CPPs disclosed herein may include AA_{H1}-D-Arg or D-Arg-AA_{H1}. In other embodiments, the CPPs disclosed herein may include AA_{H1}-L-Arg or L-Arg-AA_{H1}. In some embodiments, the presence of the hydrophobic amino acid on the N- or C-terminus of the D-Arg or L-Arg, or a combination thereof, in the CPP improves the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In some embodiments, the presence of the hydrophobic amino acid on the N- and/or C-terminus of the D-Arg and/or L-Arg in the CPP improves the cytosolic uptake efficiency by about 20 fold.

The size of the hydrophobic amino acid on the N- or C-terminus of the D-Arg or an L-Arg, or a combination thereof (i.e., AA_{H1}), may be selected to improve cytosolic delivery efficiency of the CPP. For example, a larger hydrophobic amino acid on the N- or C-terminus of a D-Arg or L-Arg, or a combination thereof, improves cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. In some embodiments, the size of the hydrophobic amino acid is measured in terms of the molecular weight of the hydrophobic amino acid, and the larger hydrophobic amino acid has a side chain with a molecular weight of at least about 90 g/mol, or at least about 130 g/mol, or at least about 141 g/mol. In particular embodiments, the size of the amino acid is measured in terms of the SASA of the hydrophobic side chain, and the larger hydrophobic amino acid has a side chain with a SASA greater than that of alanine, or greater than that of glycine. In other embodiments, AA_{H1} has a hydrophobic side chain with a SASA greater than or equal to about piperidine-2-carboxylate, greater than or equal to about tryptophan, greater than or equal to about phenylalanine, or equal to or greater than about naphthylalanine. In some embodiments, AA_{H1} and AAH₂ independently have a side with a SASA in the range of from about 200 Å² to about 1000 Å2, e.g, about 250 Å², 300 Å², 350 Å², 400 Å², 450 Å², 500 Å², 550 Å², 650 Å², 700 Å², 750 Å², 800 Å², 850 Å², 900 Å², and about 950 Å², inclusive of all values and subranges therebetween.

In some embodiments, AA_{H1} has a side chain with a SASA of at least about 200 Å², at least about 210 Å2, at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². In some embodiments, AAH₂ has a side chain side with a SASA of at least about 200 Å2, at least about 210 Å2, at least about 220 Å2, at least about 240 Å2, at least about 250 Å2, at least about 260 Å2, at least about 270 Å2, at least about 280 Å2, at least about 290 Å2, at least about 300 Å2, at least about 310 Å2, at least about 320 Å2, or at least about 330 Å2. In some embodiments, the side chains of AA_{H1} and AA_{H2} have a combined SASA of at least about 350 Å2, at least about 360 Å2, at least about 370 Å², at least about 380 Å₂, at least about 390 Å², at least about 400 Å2, at least about 410 Å², at least about 420 Å², at least about 430 Å², at least about 440 Å², at least about 450 Å², at least about 460 Å², at least about 470 Å², at least about 480 Å², at least about 490 Å², greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å2, at least about 620 Å², at least about 630 Å², at least about 640 Å², greater than about 650 Å2, at least about 660 Å2, at least about 670 Å2, at least about 680 Å2, at least about 690 Å2, or at least about 700 Å2. In some embodiments, AA_{H2} is a hydrophobic amino acid with a side chain having a SASA that is less than or equal to the SASA of the hydrophobic side chain of AA_{H1}. By way of example, and not by limitation, a CPP having a Nal-Arg motif exhibits improved cytosolic delivery efficiency compared to an otherwise identical CPP having a Phe-Arg motif; a CPP having a Phe-Nal-Arg motif exhibits improved cytosolic delivery efficiency compared to an otherwise identical CPP having a Nal-Phe-Arg motif; and a phe-Nal-Arg motif exhibits improved cytosolic delivery efficiency compared to an otherwise identical CPP having a nal-Phe-Arg motif. In some embodiments, the presence of the larger hydrophobic amino acid on the N- or C-terminus of the D-Arg or L-Arg, or a combination thereof, in the CPP improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween. In particular embodiments, the presence of the larger hydrophobic amino acid on the N- and/or C-terminus of the D-Arg and/or L-Arg in the CPP improves the cytosolic uptake efficiency by about 20 fold.

As used herein, “hydrophobic surface area” or “SASA” refers to the surface area (reported as square Ångstroms; Å²) of an amino acid side chain that is accessible to a solvent. In particular embodiments, SASA is calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. In certain embodiments, the SASA values described herein are based on the theoretical values listed in Table 3 below, as reported by Tien, et al. (PLOS ONE 8(11): e80635. https://doi.org/10.1371/journal.pone.0080635), which is herein incorporated by reference in its entirety for all purposes.

TABLE 3

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

The chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine (e.g., AA_{H1}, AAᵤ or AA_{z}) has the same or opposite chirality as the adjacent arginine. In some embodiments, AA_{H1} has the same chirality as the adjacent arginine. For example, when the arginine is D-arg (i.e. “r”), AA_{H1} is a D-AA_{H1}, and when the arginine is L-Arg (i.e., “R”), AA_{H1} is a L-AA_{H1}. Accordingly, in some embodiments, the CPPs disclosed herein may include at least one of the following motifs: D-AA_{H1}-D-arg, D-arg-D-AA_{H1}, L-AA_{H1}-L-Arg, or L-Arg-LAAH1. In some embodiments, the presence of the hydrophobic amino acid having the same chirality as the adjacent arginine improves cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold inclusive of all values and subranges therebetween. In some embodiments, the presence of the hydrophobic amino acid having the same chirality as the adjacent arginine improves the cytosolic uptake efficiency by about 2.5 fold.

As discussed above, the disclosure provides for various modifications to a cyclic peptide sequence which may improve cytosolic delivery efficiency. In some embodiments, improved cytosolic uptake efficiency can be measured by comparing the cytosolic delivery efficiency of the CPP having the modified sequence to a proper control sequence. In some embodiments, the control sequence does not include a particular modification (e.g., matching chirality of R and AA_{H1}) but is otherwise identical to the modified sequence. In other embodiments, the control has the following sequence: cyclic (FΦRRRRQ)

As used herein cytosolic delivery efficiency refers to the ability of a CPP to traverse a cell membrane and enter the cytosol. In some embodiments, cytosolic delivery efficiency of the CPP is not dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-cargo conjugate) over the concentration of the cCPP (or the cCPP-cargo conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FTIC dye) and measuring the fluorescence intensity using techniques well-known in the art.

In particular embodiments, relative cytosolic delivery efficiency is determined by comparing (i) the amount of a cCPP of the invention internalized by a cell type (e.g., HeLa cells) to (ii) the amount of the control cCPP internalized by the same cell type. To measure relative cytosolic delivery efficiency, the cell type may be incubated in the presence of a cCPP of the invention for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

In other embodiments, relative cytosolic delivery efficiency can be determined by measuring the IC₅₀ of a cCPP having a modified sequence for an intracellular target, and comparing the IC₅₀ of the cCPP having the modified sequence to a proper control sequence (as described herein).

In some embodiments, the relative cytosolic delivery efficiency of the CPPs described herein is in the range of from about 50% to about 450% compared to cyclo(FΦRRRRQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. In other embodiments, the relative cytosolic delivery efficiency of the CPPs described herein is improved by greater than about 600% compared to cyclo(FΦRRRRQ).

In other embodiments, the absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

In some embodiments, the cCPPs disclosed herein (e.g., Formulae I, I-A to I-F, and Formula IV-A to IV-F) are selected from the sequences provided in Table 4 below.

TABLE 4

| | | | | | Cellular Entry | |
|---|---|---|---|---|---|---|
| | | SEQ. | | | Efficiency^{b} | |
| | | ID. | Molecular Mass | | In 10% | In 1% |
| Peptide | Sequence^{a} | NO. | Calcd | Obsd | FBS | FBS |
| CPP1 | cyclo(FΦRRRRQ) | 58 | 1827.86 | 1828.837 | 21 | |
| CPP9 | cyclo(fΦRrRrQ) | 59 | 1827.86 | 1828.778 | 94 ± 7 | 32 ± 6 |
| CPP12 | cyclo(FfΦRrRrQ) | 60 | 1974.93 | 1975.839 | 100 | 100 |
| CPP1-1 | cyclo(F^{tBu}RRRRQ) | 61 | 1686.84 | 1687.868 | 6.7 ± 0.4 | |
| CPP1-2 | cyclo(Dap^{Hexan}RRRRQ) | 62 | 1667.83 | 1668.816 | 2.9 ± 0.2 | |
| CPP1-3 | cyclo(Dap^{Octan}RRRRQ) | 63 | 1696.86 | 1696.889 | 5.7 ± 0.6 | |
| CPP1-4 | cyclo(Dap^{Deca}RRRRQ) | 64 | 1723.89 | 1746.906 | 57 ± 19 | |
| CPP1-5 | cyclo(Dap^{1-Pyren}RRRRQ) | 65 | 1797.82 | 1798.836 | 10 ± 20 | |
| CPP1-6 | cyclo(Dap^{3,3-dipheny}RRRRQ) | 66 | 1706.81 | 1707.834 | 3.9 ± 0.4 | |
| CPP1-7 | cyclo(Dap^{Fmoc}RRRRQ) | 67 | 1791.83 | 1792.861 | 17 ± 1 | |
| CPP1-8 | cyclo(Dap^{1-Pyreneb}RRRRQ) | 68 | 1839.86 | 1840.907 | 24 ± 1 | |
| CPP1-9 | Not synthesized | | | | | |
| CPP1-10 | cyclo(Dap^{Deca}RrRrQ) | 69 | 1723.89 | 1724.923 | 21 ± 9 | 24 |
| CPP1-11 | cyclo(Dap^{Deca}rRrRQ) | 70 | 1723.89 | 1724.895 | 20 ± 6 | 29 |
| CPP1-12 | cyclo(Dap^{Deca}ARRRQ) | 71 | 1638.83 | 1639.834 | 26 ± 4 | 34 |
| CPP1-13 | cyclo(Dap^{Deca}RRRAQ) | 72 | 1638.83 | 1639.824 | 32 ± 2 | 33 |
| CPP1-14 | cyclo(Dap^{Deca}RRRRRQ) | 73 | 1880.00 | 1880.979 | 12 ± 1 | 21 |
| CPP1-15 | cyclo(Lys^{Deca}RRRRQ) | 74 | 1765.94 | 1767.638 | 76 ± 21 | 44 ± 11 |
| CPP1-16 | cyclo(Dap^{Deca}RRRQ) | 75 | 1567.79 | 1568.692 | 3.0 ± 0.1 | 25 |
| CPP1-17 | cyclo(Orn^{Deca}RRRRQ) | 76 | 1751.93 | 1752.832 | 115 ± 32 | 58 ± 22 |
| CPP1-18 | cyclo(Lys^{Deca}RrRrQ) | 77 | 1765.94 | 1766.908 | 31 | |
| CPP1-19 | cyclo(Lys^{Deca}rRrRQ) | 78 | 1765.94 | 1766.880 | 12 | |
| CPP1-20 | cyclo(Asp^{Decy}RRRRQ) | 79 | 1737.91 | 1738.830 | 281 ± 59 | 94 ± 41 |
| CPP1-22 | cyclo(Asp^{Decy}RrRrQ) | 80 | 1737.91 | 1738.844 | 11 | |
| CPP1-23 | cyclo(Glu^{Decy}RrRrQ) | 81 | 1751.93 | 1752.833 | 1.4 | |
| CPP1-24 | cyclo(Asp^{Decy}rRrRQ) | 82 | 1737.91 | 1738.854 | 8.6 | |
| CPP1-25 | cyclo(Glu^{Decy}rRrRQ) | 83 | 1751.93 | 1752.837 | 34 | |

^{a}Single-letter codes for amino acids. Φ, 2-naphthylalanine. tBu = tert-butanoyl, hexan = hexanoyl, octan = octanoyl, deca = decanoyl, 1-pyren = pyrenol, 3,3-diphenyl = 3,3-diphenoyl, Fmoc = fluorenylmethyloxycarbonoyl, 1-pyreneb = 1-pyrenylbutanoyl, decy = decynoyl
^{b}All values are relative to that of CPP12 (100%).

In particular embodiments, the cCCPs disclosed herein have the following structure:

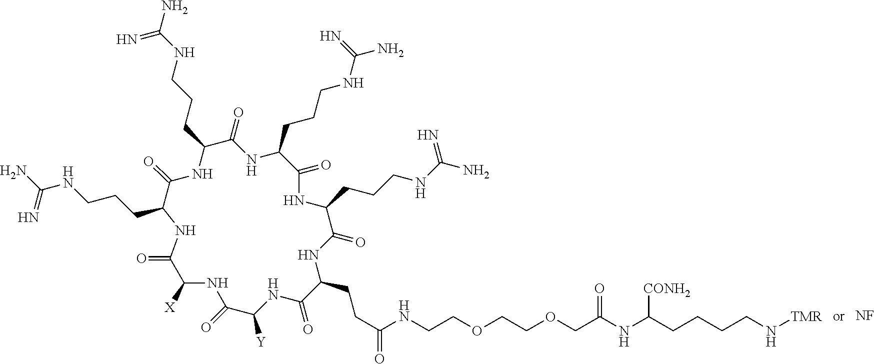

wherein X is selected from:

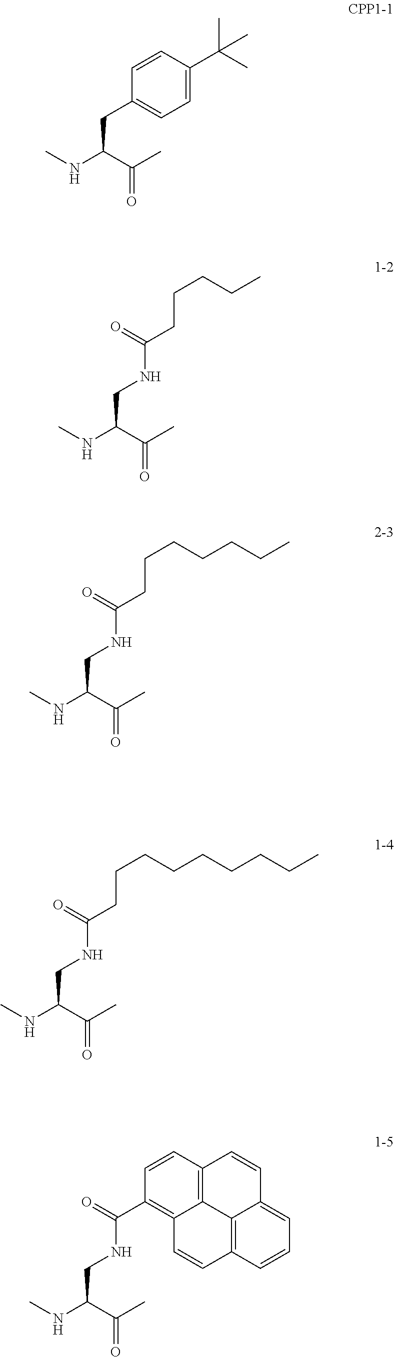

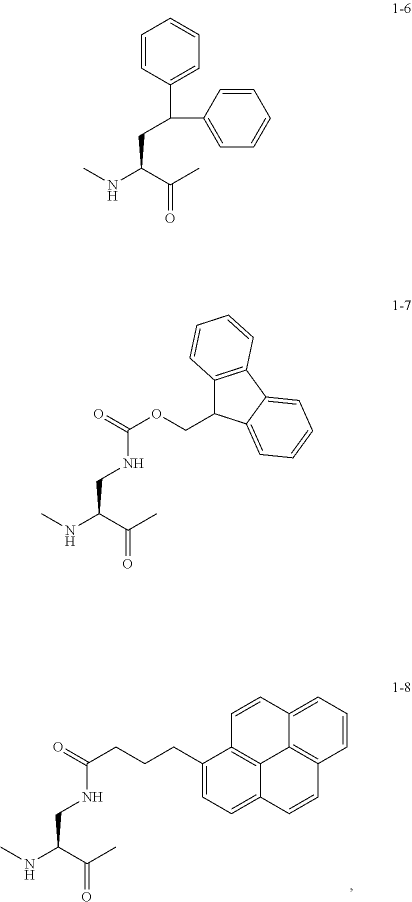 [SMILES: CN[C@@H](CC(c1ccccc1)c1ccccc1)C(C)=O ; CN[C@@H](CNC(=O)OCC1c2ccccc2-c2ccccc21)C(C)=O ; CN[C@@H](CNC(=O)CCCc1ccc2ccc3cccc4ccc1c2c34)C(C)=O]

wherein the amino acid bearing Y is absent.

In some embodiments, the cCPPs disclosed herein (e.g., Formulae I, I-A to I-F, IV-A to IV-F) are selected from:

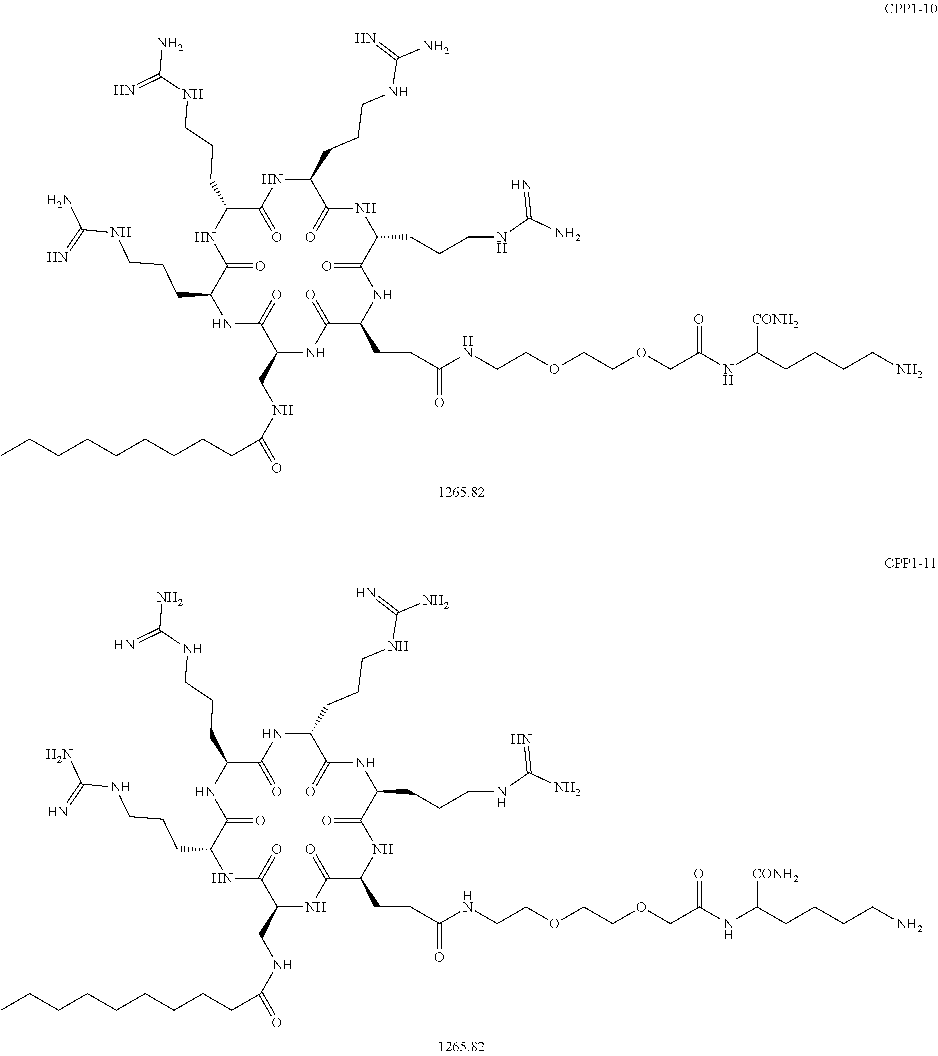 [SMILES: CCCCCCCCCC(=O)NC[C@@H]1NC(=O)[C@H](CCC(=O)NCCOCCOCC(=O)NC(CCCCN)C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O ; CCCCCCCCCC(=O)NC[C@@H]1NC(=O)[C@H](CCC(=O)NCCOCCOCC(=O)NC(CCCCN)C(N)=O)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC1=O]

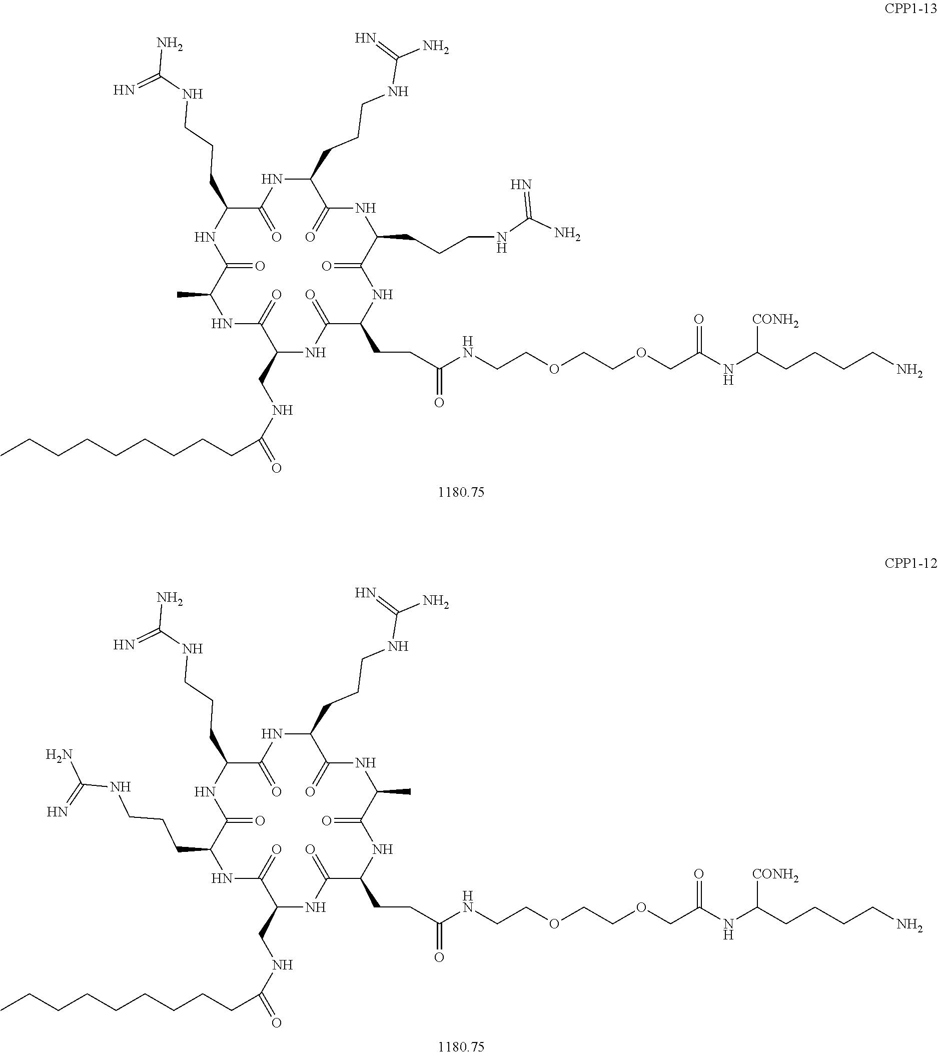 [SMILES: CCCCCCCCCC(=O)NC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](C)NC1=O ; CCCCCCCCCC(=O)NC[C@@H]1NC(=O)[C@H](CCC(=O)NCCOCCOCC(=O)NC(CCCCN)C(N)=O)NC(=O)[C@H](C)NC(=O)[C@H](CCCNC(=N)N)NC(=O)[C@H](CCCNC(=N)N)NC(=O)[C@H](CCCNC(=N)N)NC1=O]

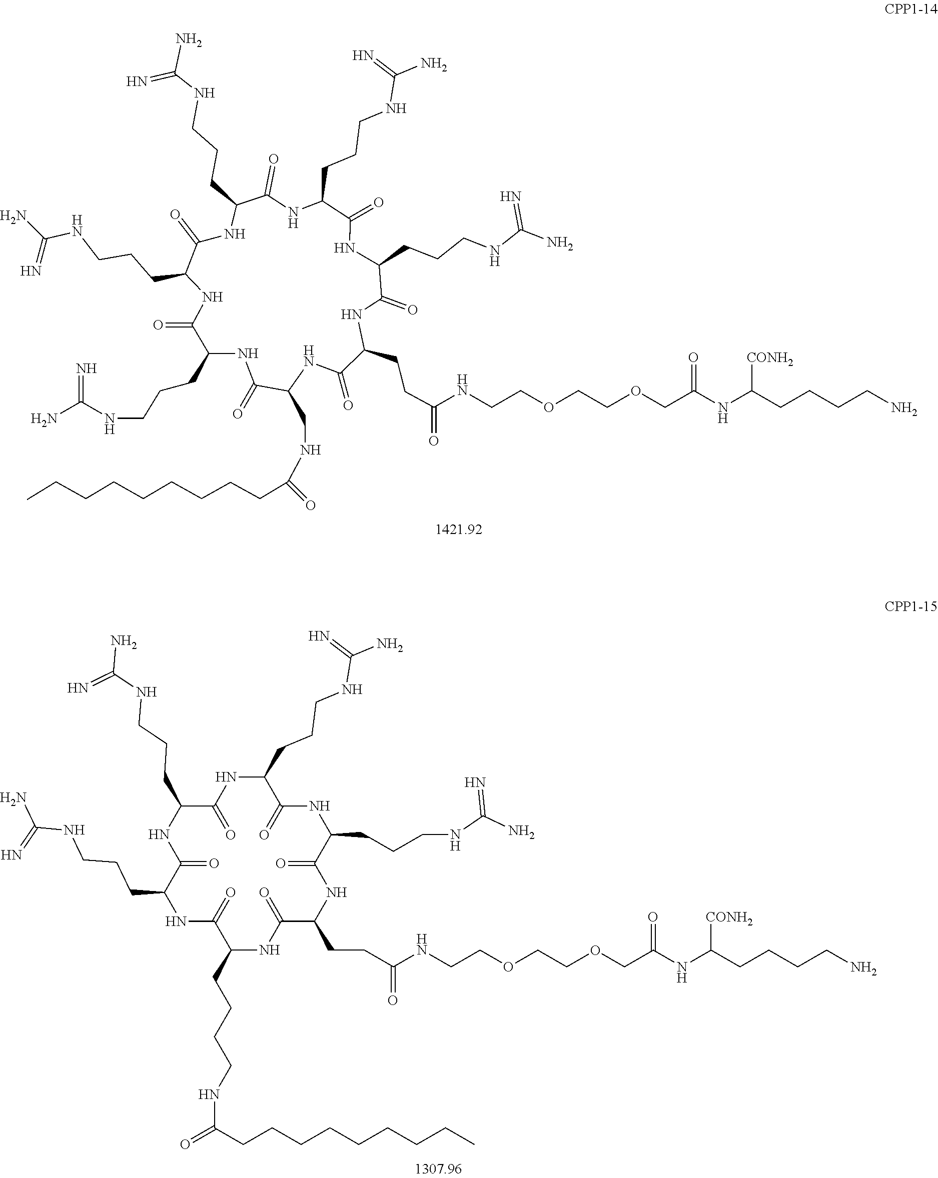 [SMILES: CCCCCCCCCC(=O)NC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC1=O ; CCCCCCCCCC(=O)NCCCC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC1=O]

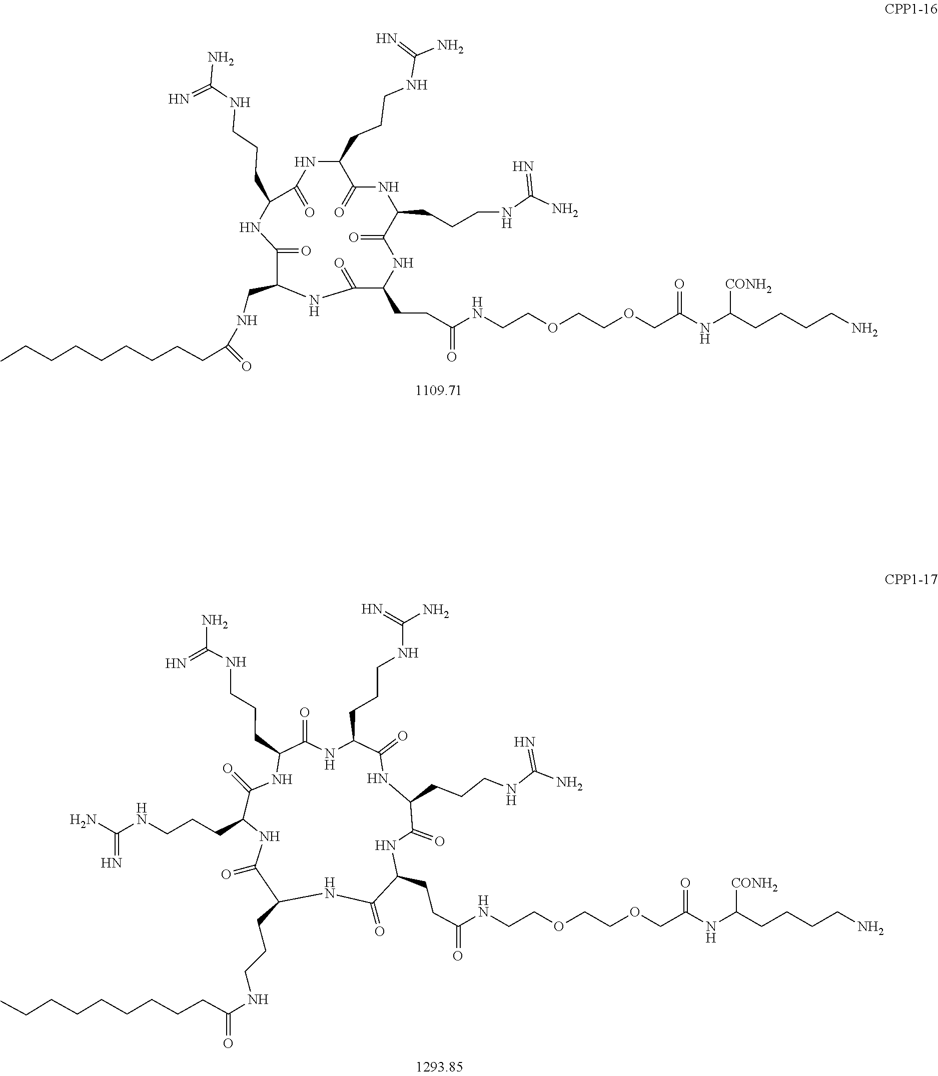 [SMILES: CCCCCCCCCC(=O)NC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC1=O ; CCCCCCCCCC(=O)NCCC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC1=O]

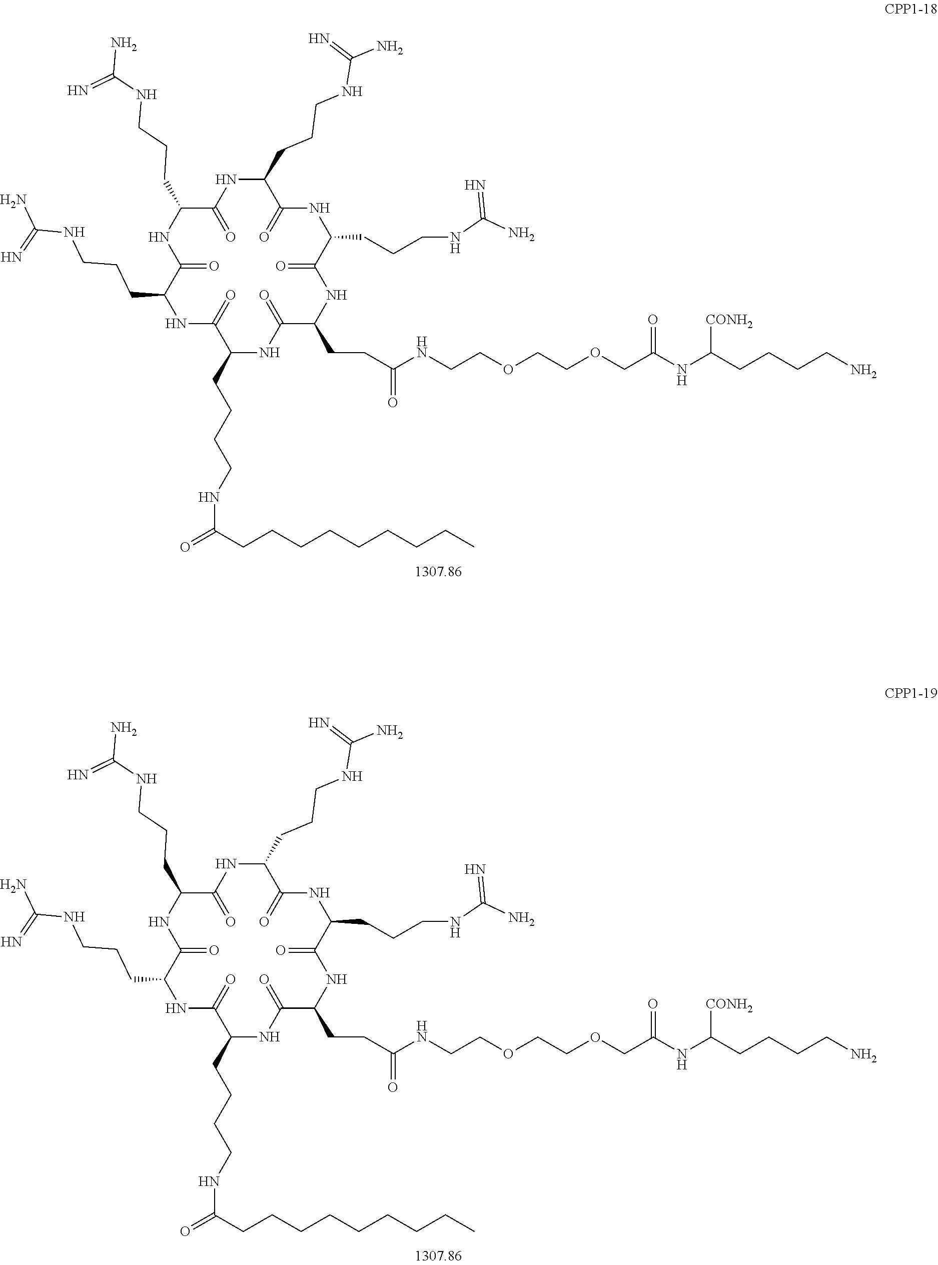 [SMILES: CCCCCCCCCC(=O)NCCCC[C@@H]1NC(=O)[C@H](CCC(=O)NCCOCCOCC(=O)NC(CCCCN)C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O ; CCCCCCCCCC(=O)NCCCC[C@@H]1NC(=O)[C@H](CCC(=O)NCCOCCOCC(=O)NC(CCCCN)C(N)=O)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC1=O]

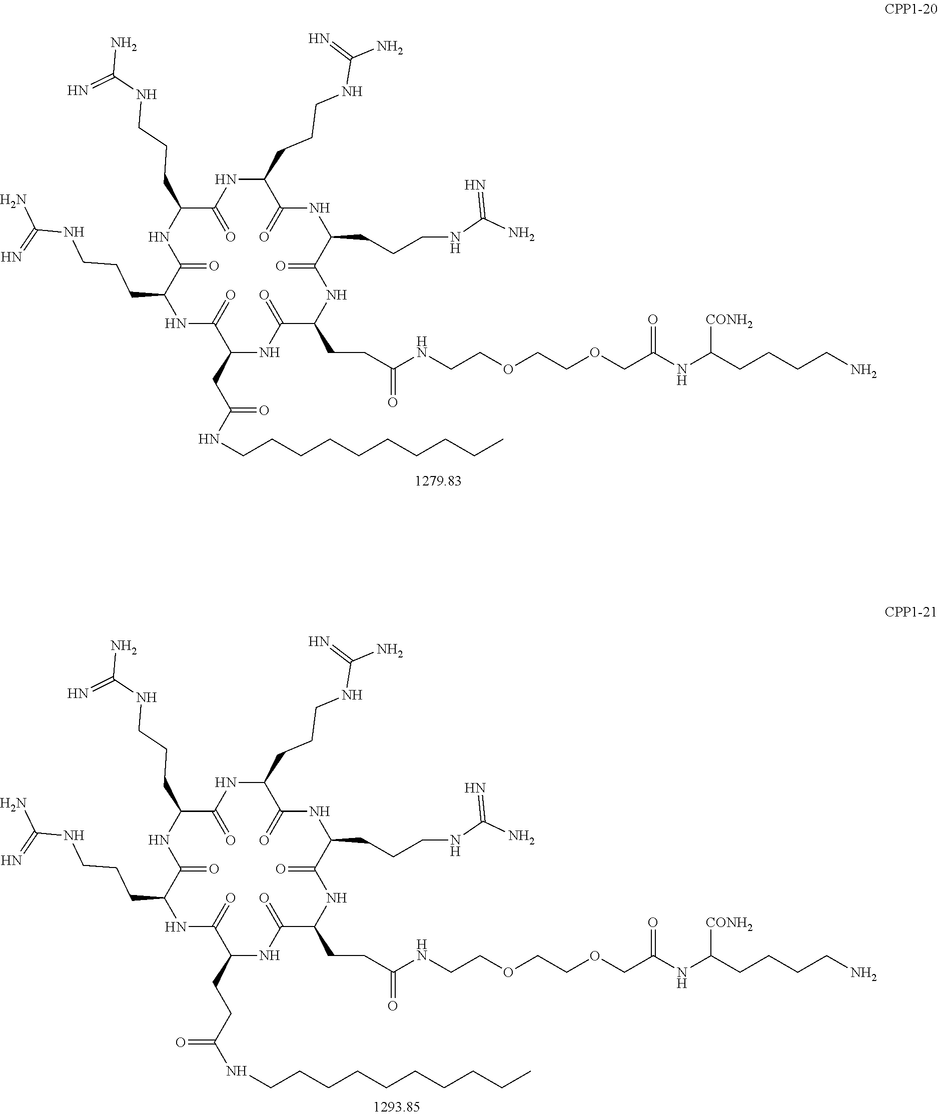 [SMILES: CCCCCCCCCCNC(=O)C[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC1=O ; CCCCCCCCCCNC(=O)CC[C@@H]1NC(=O)[C@H](CCC(=O)NCCOCCOCC(=O)NC(CCCCN)C(N)=O)NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC1=O]

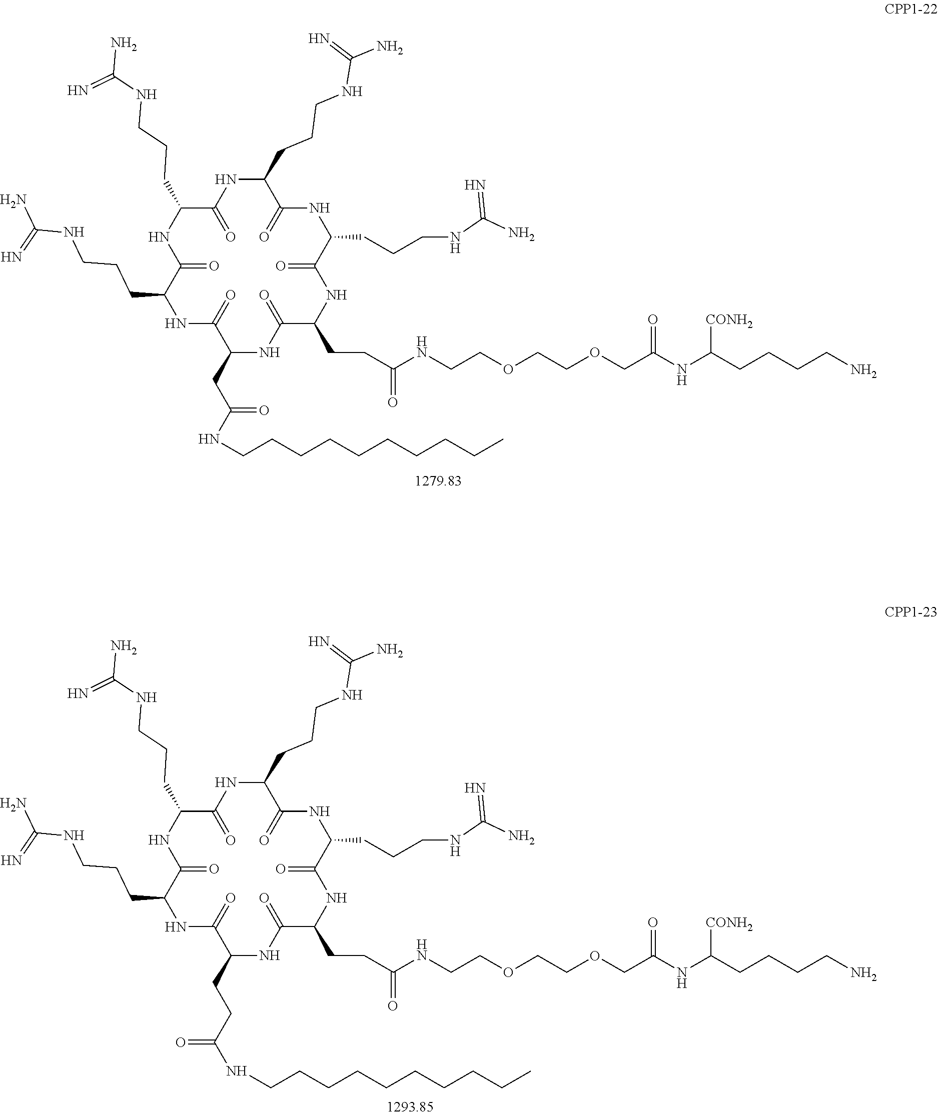 [SMILES: CCCCCCCCCCNC(=O)C[C@@H]1NC(=O)[C@H](CCC(=O)NCCOCCOCC(=O)NC(CCCCN)C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O ; CCCCCCCCCCNC(=O)CC[C@@H]1NC(=O)[C@H](CCC(=O)NCCOCCOCC(=O)NC(CCCCN)C(N)=O)NC(=O)[C@@H](CCCNC(=N)N)NC(=O)[C@H](CCCNC(=N)N)NC(=O)[C@@H](CCCNC(=N)N)NC(=O)[C@H](CCCNC(=N)N)NC1=O]

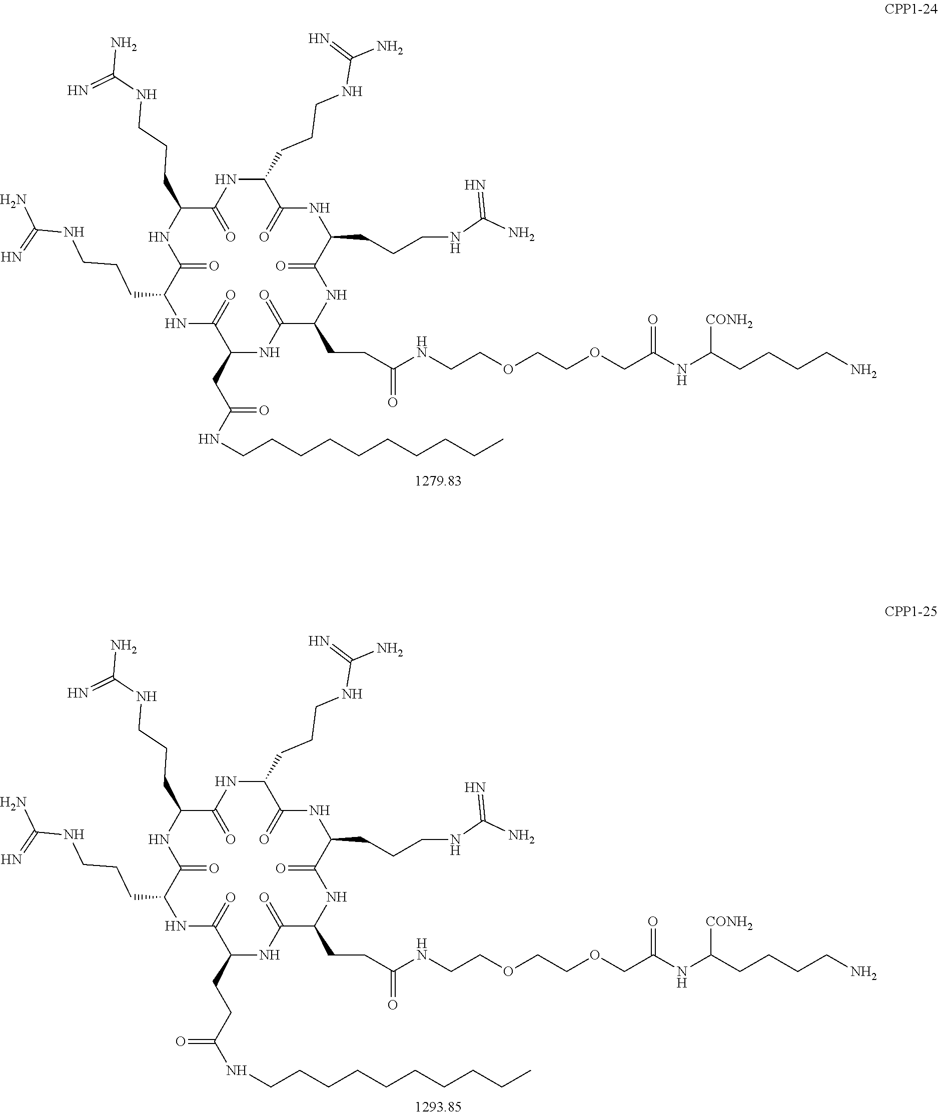 [SMILES: CCCCCCCCCCNC(=O)C[C@@H]1NC(=O)[C@H](CCC(=O)NCCOCCOCC(=O)NC(CCCCN)C(N)=O)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC1=O ; CCCCCCCCCCNC(=O)CC[C@@H]1NC(=O)[C@H](CCC(=O)NCCOCCOCC(=O)NC(CCCCN)C(N)=O)NC(=O)[C@H](CCCNC(=N)N)NC(=O)[C@@H](CCCNC(=N)N)NC(=O)[C@H](CCCNC(=N)N)NC(=O)[C@@H](CCCNC(=N)N)NC1=O]

### Cargo

In some embodiments, the CPPs disclosed herein can further include a cargo moiety, which may comprise a peptide. The cargo moiety can comprise one or more detectable moieties, one or more therapeutic moieties, one or more targeting moieties, or any combination thereof. In some embodiments, the cargo moiety may be a peptide sequence or a non-peptidyl therapeutic agent. In some embodiments, the cargo moiety can be coupled to an amino group (e.g., N-terminus), a carboxylate group (e.g., C-terminus), or a side chain of one or more amino acids in the cCPP. In some embodiments, the cCPP and the cargo moiety together are cyclic (referred to herein as “endocyclic”). In the endocyclic system, the cargo moiety may be located between amino acids of the cCPP. In some embodiments, the cargo moiety independently forms a peptide bond with each of the adjacent amino acids. In some embodiments, the cCPP is cyclic and the cargo moiety is appended to the cyclic cell penetrating peptide moiety structure (referred to herein as “exocyclic”). In some embodiments, the cargo moiety is cyclic and the cCPP is cyclic, and together they form a bicyclic system (referred to herein as “bicyclic”).

In some embodiments, the cCPP further comprises a linker group (“L”), and the cargo is attached to the linker group, forming a bicyclic cCPP and cargo moiety. In certain embodiments, the cCPP further comprises a linker group (“L”), and cargo is attached to the linker group and a side chain of an amino acid of the CPP, forming a bicyclic cCPP and cargo moiety. Examples of linkers used to form a bicyclic include trimesic acid or nitrilotriacetic acid, as described in U.S. Patent App. Pub. 2016/0115202, which is herein incorporated by reference in its entirety for all purposes.

It is also disclosed herein that for the endocyclic structure, some amino acids in the CPP can also be part of the cargo moity. For example, a peptide pentrating moiety FNalRR can be formed from FNal and a cargo moiety comprising two Args. In this case, the two Arg residues perform dual functions. Thus, in some cases the sequence of the cargo moiety is taken into account when referring to the peptide penetrating moiety.

### Cargo Moiety

The cargo moiety can comprise any cargo of interest, for example a linker moiety, a detectable moiety, a therapeutic moiety, a targeting moiety, and the like, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

In some examples the cargo moiety can comprise any of those listed in Table 5, or derivatives or combinations thereof.

TABLE 5
Example cargo moieties

| SEQ ID NO | Abbreviation | Sequence* |
|---|---|---|
| 1 | R₅ | RRRRR |
| 2 | A₅ | AAAAA |
| 3 | F₄ | FFFF |
| 4 | PCP | DE(pCAP)LI |
| 5 | A₇ | AAAAAAA |
| 6 | | RARAR |
| 7 | | DADAD |
| 8 | | DΩUD |
| 9 | | UTRV |
| 10 | | D-pThr-Pip-Nal |

*pCAP, phosphocoumaryl amino propionic acid; Ω, norleucine; U, 2-aminobutyric acid; D-pThr is D-phosphothreonine, Pip is L-piperidine-2-carboxylate.

### Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, a isotope detectable spin resonance label, a paramagnetic moiety, a chromophore, or any combination thereof. In some embodiments, the label is detectable without the addition of further reagents.

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins; benzoporphyrins; azabenzoporphyrine; napthoporphyrin; phthalocyanine; polycyclic aromatic hydrocarbons such as perylene, perylene diimine, pyrenes; azo dyes; xanthene dyes; boron dipyoromethene, aza-boron dipyoromethene, cyanine dyes, metal-ligand complex such as bipyridine, bipyridyls, phenanthroline, coumarin, and acetylacetonates of ruthenium and iridium; acridine, oxazine derivatives such as benzophenoxazine; aza-annulene, squaraine; 8-hydroxyquinoline, polymethines, luminescent producing nanoparticle, such as quantum dots, nanocrystals; carbostyril; terbium complex; inorganic phosphor; ionophore such as crown ethers affiliated or derivatized dyes; or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra(pentafluorophenyl)porphyrin; Pt (II) meso-tetra(pentafluorophenyl) porphyrin; Ru (II) tris(4,7-diphenyl-1,10-phenanthroline) (Ru (dpp)₃); Ru (II) tris(1,10-phenanthroline) (Ru(phen)₃), tris(2,2′-bipyridine)rutheniurn (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitirophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5(6)-carboxy-fluorecsein; carboxynaphthofluorescein; 8-hydroxypyrene-1,3,6-trisulfonic acid; semi-naphthorhodafluor; semi-naphthofluorescein; tris(4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxycarbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcourmarin (Amc), green fluorescent protein (GFP), or derivatives or combinations thereof.

The detectible moiety can be attached to the cell penetrating peptide moiety at the amino group, the carboxylate group, or the side chain of any of the amino acids of the cell penetrating peptide moiety (e.g., at the amino group, the carboxylate group, or the side chain of any amino acid in the CPP).

### Therapeutic Moiety

The disclosed compounds can also comprise a therapeutic moiety. In some examples, the cargo moiety comprises a therapeutic moiety. The detectable moiety can be linked to a therapeutic moiety or the detectable moiety can also serve as the therapeutic moiety. Therapeutic moiety refers to a group that when administered to a subject will reduce one or more symptoms of a disease or disorder.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

In some examples, the therapeutic moiety can comprise an antiviral agent, such as ganciclovir, azidothymidine (AZT), lamivudine (3TC), etc.

In some examples, the therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some examples, the therapeutic moiety can comprise an anti-inflammatory agent.

In some examples, the therapeutic moiety can comprise dexamethasone (Dex).

In other examples, the therapeutic moiety comprises a therapeutic protein. For example, some people have defects in certain enzymes (e.g., lysosomal storage disease). It is disclosed herein to deliver such enzymes/proteins to human cells by linking to the enzyme/protein to one of the disclosed cell penetrating peptides. The disclosed cell penetrating peptides have been tested with proteins (e.g., GFP, PTP1B, actin, calmodulin, troponin C) and shown to work.

In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease, such as cancer, cystic fibrosis, diabetes, obesity, or combinations thereof. For example, the targeting moiety can comprise any of the sequences listed in Table 6.

TABLE 6
Example targeting moieties

| SEQ ID | | |
|---|---|---|
| NO | Abbreviation* | Sequence |
| 11 | PΘGΛYR | Pro-Pip-Gly-F₂Pmp-Tyr-Arg |
| 12 | SΘIΛΛR | Ser-Pip-Ile-F₂Pmp-F₂Pmp-Arg |
| 13 | IHIΛIR | Ile-His-Ile-F₂Pmp-Ile-Arg |
| 14 | AaIΛΘR | Ala-(D-Ala)-Ile-F₂Pmp-Pip-Arg |
| 15 | ΣSΘΛvR | Fpa-Ser-Pip-F₂Pmp-(D-Val)-Arg |
| 16 | ΘnPΛAR | Pip-(D-Asn)-Pro-F₂Pmp-Ala-Arg |
| 17 | TΨAΛGR | Tyr-Phg-Ala-F₂Pmp-Gly-Arg |
| 18 | AHIΛaR | Ala-His-Ile-F₂Pmp-(D-Ala)-Arg |
| 19 | GnGΛpR | Gly-(D-Asn)-Gly-F₂Pmp-(D-Pro)-Arg |
| 20 | fQΘΛIR | (D-Phe)-Gln-Pip-F₂Pmp-Ile-Arg |
| 21 | SPGΛHR | Ser-Pro-Gly-F₂Pmp-His-Arg |
| 22 | ΘYIΛHR | Pip-Tyr-Ile-F₂Pmp-His-Arg |
| 23 | SvPΛHR | Ser-(D-Val)-Pro-F₂Pmp-His-Arg |
| 24 | AIPΛnR | Ala-Ile-Pro-F₂Pmp-(D-Asn)-Arg |
| 25 | ΣSIΛQF | Fpa-Ser-Ile-F₂Pmp-Gln-Arg |
| 26 | AaΨΛfR | Ala-(D-Ala)-Phg-F₂Pmp-(D-Phe)-Arg |
| 27 | ntΨΛΨR | (D-Asn)-(D-Thr)-Phg-F₂Pmp-Phg-Arg |
| 28 | IPΨΛΩR | Ile-Pro-Phg-F₂Pmp-Nle-Arg |
| 29 | QΘΣΛΘR | Gln-Pip-Fpa-F₂Pmp-Pip-Arg |
| 30 | nAΣΛGR | (D-Asn)-Ala-Fpa-F₂Pmp-Gly-Arg |
| 31 | ntYΛAR | (D-Asn)-(D-Thr)-Tyr-F₂Pmp-Ala-Arg |
| 32 | eAΨΛvR | (D-Glu)-Ala-Phg-F₂Pmp-(D-Val)-Arg |
| 33 | IvΨΛAR | Ile-(D-Val)-Phg-F₂Pmp-Ala-Arg |
| 34 | YtΨΛAR | Tyr-(D-Thr)-Phg-F₂Pmp-Ala-Arg |
| 35 | nΘΨΛIR | (D-Asn)-Pip-Phg-F₂Pmp-Ile-Arg |
| 36 | ΘnWΛHR | Pip-(D-Asn)-Trp-F₂Pmp-His-Arg |
| 37 | YΘvΛIR | Tyr-Pip-(D-Val)-F₂Pmp-Ile-Arg |
| 38 | nSAΛGR | (D-Asn)-Ser-(D-Ala)-F₂Pmp-Gly-Arg |
| 39 | tnvΛaR | (D-Thr)-(D-Asn)-(D-Val)-F₂Pmp-(D-Ala)-Arg |
| 40 | ntvΛtR | (D-Asn)-(D-Thr)-(D-Val)-F₂Pmp-(D-Thr)-Arg |
| 41 | SItΛYR | Ser-Ile-(D-Thr)-F₂Pmp-Tyr-Arg |
| 42 | nΣnΛ1R | (D-Asn)-Fpa-(D-Asn)-F₂Pmp-(D-Leu)-Arg |
| 43 | YnnΛΩR | Tyr-(D-Asn)-(D-Asn)-F₂Pmp-Nle-Arg |
| 44 | nYnΛGR | (D-Asn)-Tyr-(D-Asn)-F₂Pmp-Gly-Arg |
| 45 | AWnΛAR | Ala-Trp-(D-Asn)-F₂Pmp-Ala-Arg |
| 46 | vtHΛYR | (D-Val)-(D-Thr)-His-F₂Pmp-Tyr-Arg |
| 47 | PΨHΛΘR | Pro-Phg-His-F₂Pmp-Pip-Arg |
| 48 | nΨHΛGR | (D-Asn)-Phg-His-F₂Pmp-Gly-Arg |
| 49 | PAHΛGR | Pro-Ala-His-F₂Pmp-Gly-Arg |
| 50 | AYHΛIR | Ala-Tyr-His-F₂Pmp-Ile-Arg |
| 51 | nΘeΛYR | (D-Asn)-Pip-(D-Glu)-F₂Pmp-Tyr-Arg |
| 52 | vSSΛtR | (D-Val)-Ser-Ser-F₂Pmp-(D-Thr)-Arg |
| 53 | aΞt′ϑΦ′YNK | ((D-Ala)-Sar-(D-pThr)-Pp-Nal-Tyr-Gln)-Lys |
| 54 | Tm(aΞt′ϑΦ′RA)Dap | Tm((D-Ala)-Sar-(D-pThr)-Pp-Nal-Arg-Ala)-Dap |
| 55 | Tm(aΞt′ϑΦ′RAa)Dap | Tm((D-Ala)-Sar-(D-pThr)-Pp-Nal-Arg-Ala-(D- |
| | | Ala))-Dap |
| 56 | Tm(aΞtϑΦ′RAa)Dap | Tm((D-Ala)-Sar-(D-Thr)-Pp-Nal-Arg-Ala-(D- |
| | | Ala))-Dap |
| 57 | Tm(aΞtaΦ′RAa)Dap | Tm((D-Ala)-Sar-(D-Thr)-(D-Ala)-Nal-Arg-Ala- |
| | | (D-Ala))-Dap |

*Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ′: L-β-naphthylalanine; Pp, ϑ: L-pipecolic acid; Sar, Ξ: sarcosine; Tm, trimesic acid.

The targeting moiety and cell penetrating peptide moiety can overlap. That is, the residues that form the cell penetrating peptide moiety can also be part of the sequence that forms the targeting moiety, and vice a versa.

The therapeutic moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the therapeutic moiety can be attached to the detectable moiety.

In some examples, the therapeutic moiety can comprise a targeting moiety that can act as an inhibitor against Ras (e.g., K-Ras), PTP1B, Pin1, Grb2 SH2, CAL PDZ, and the like, or combinations thereof.

Ras is a protein that in humans is encoded by the RAS gene. The normal Ras protein performs an essential function in normal tissue signaling, and the mutation of a Ras gene is implicated in the development of many cancers. Ras can act as a molecular on/off switch, once it is turned on Ras recruits and activates proteins necessary for the propagation of growth factor and other receptors' signal. Mutated forms of Ras have been implicated in various cancers, including lung cancer, colon cancer, pancreatic cancer, and various leukemias.

Protein-tyrosine phosphatase 1B (PTP1B) is a prototypical member of the PTP superfamily and plays numerous roles during eukaryotic cell signaling. PTP1B is a negative regulator of the insulin signaling pathway, and is considered a promising potential therapeutic target, in particular for the treatment of type II diabetes. PIP1B has also been implicated in the development of breast cancer.

Pin1 is an enzyme that binds to a subset of proteins and plays a role as a post phosphorylation control in regulating protein function. Pin1 activity can regulate the outcome of proline-directed kinase signaling and consequently can regulate cell proliferation and cell survival. Deregulation of Pin1 can play a role in various diseases. The up-regulation of Pin1 may be implicated in certain cancers, and the down-regulation of Pin1 may be implicated in Alzheimer's disease. Inhibitors of Pin1 can have therapeutic implications for cancer and immune disorders.

Grb2 is an adaptor protein involved in signal transduction and cell communication. The Grb2 protein contains one SH2 domain, which can bind tyrosine phosphorylated sequences. Grb2 is widely expressed and is essential for multiple cellular functions. Inhibition of Grb2 function can impair developmental processes and can block transformation and proliferation of various cell types.

It was recently reported that the activity of cystic fibrosis membrane conductance regulator (CFTR), a chloride ion channel protein mutated in cystic fibrosis (CF) patients, is negatively regulated by CFTR-associated ligand (CAL) through its PDZ domain (CAL-PDZ) (Wolde, M et al. J. Biol. Chem. 2007, 282, 8099). Inhibition of the CFTR/CAL-PDZ interaction was shown to improve the activity of ΔPhe508-CFTR, the most common form of CFTR mutation (Cheng, S H et al. Cell 1990, 63, 827; Kerem, B S et al. Science 1989, 245, 1073), by reducing its proteasome-mediated degradation (Cushing, P R et al. Angew. Chem. Int. Ed. 2010, 49, 9907). Thus, disclosed herein is a method for treating a subject having cystic fibrosis by administering an effective amount of a compound or composition disclosed herein. The compound or composition administered to the subject can comprise a therapeutic moiety that can comprise a targeting moiety that can act as an inhibitor against CAL PDZ. Also, the dcompositions or compositions disclosed herein can be administered with a molecule that corrects the CFTR function.

Also disclosed herein are compositions comprising the compounds described herein.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl(trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used.

Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxyacetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxyacetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thianisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples of the methods of treating of treating cancer or a tumor in a subject, the compound or composition administered to the subject can comprise a therapeutic moiety that can comprise a targeting moiety that can act as an inhibitor against Ras (e.g., K-Ras), PTP1B, Pin1, Grb2 SH2, or combinations thereof.

The disclosed subject matter also concerns methods for treating a subject having a metabolic disorder or condition. In one embodiment, an effective amount of one or more compounds or compositions disclosed herein is administered to a subject having a metabolic disorder and who is in need of treatment thereof. In some examples, the metabolic disorder can comprise type II diabetes. In some examples of the methods of treating of treating the metabolic disorder in a subject, the compound or composition administered to the subject can comprise a therapeutic moiety that can comprise a targeting moiety that can act as an inhibitor against PTP1B. In one particular example of this method the subject is obese and the method comprises treating the subject for obesity by administering a composition as disclosed herein.

The disclosed subject matter also concerns methods for treating a subject having an immune disorder or condition. In one embodiment, an effective amount of one or more compounds or compositions disclosed herein is administered to a subject having an immune disorder and who is in need of treatment thereof. In some examples of the methods of treating of treating the immune disorder in a subject, the compound or composition administered to the subject can comprise a therapeutic moiety that can comprise a targeting moiety that can act as an inhibitor against Pin1.

The disclosed subject matter also concerns methods for treating a subject having an inflammatory disorder or condition. In one embodiment, an effective amount of one or more compounds or compositions disclosed herein is administered to a subject having an inflammatory disorder and who is in need of treatment thereof.

The disclosed subject matter also concerns methods for treating a subject having cystic fibrosis. In one embodiment, an effective amount of one or more compounds or compositions disclosed herein is administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

In some embodiments, the CPPs disclosed herein can be used for detecting or diagnosing a disease or condition in a subject. For example, a CPP can comprise a targeting moiety and/or a detectible moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly(D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

### Example 1. CPP Synthesis

Materials and General Methods. Reagents for peptide synthesis and Rink amide resin (100-200 mesh, 0.54 mmol/g) were purchased from Chem-Impex (Wood Dale, IL). The purity of the peptides was assessed by analytical HPLC and the identity was confirmed by MALDI-TOF mass spectrometric analyses on a Bruker UltrafleXtreme MALTI-TOF-TOF (Campus Chemical Instrument Center, The Ohio State University) instrument (unless noted otherwise).

Synthesis of CPP1, CPP9, CPPJ2, CPP1-1, CPP1-6. Peptides were manually synthesized on 100 mg of Rink amide resin (0.54 mmol/g) by standard Fmoc chemistry. For each cycle, the resin was swollen in DMF for 20 min and Fmoc group was then removed by treatment with 20% piperidine in DMF for 5 min (twice). The resin was washed with DMF/DCM/DMF (3 times) and the next amino acid was coupled in standard Fmoc/HATU chemistry by using Fmoc-amino acid/HATU/DIPEA (4, 4, 8 equiv) in DMF (2 h at RT). The coupling reactions were monitored by ninhydrin test after each position. After completion of the linear sequence, the allyl group on the α-carboxyl group of the C-terminal L-Glu was removed by treatment with tetrakis(triphenylphosphine)palladium/phenylsilane (0.3 and 10 equiv, respectively) in DCM for 15 min (3 times). The resin was washed by sodium dimethyldithiocarbamate dihydrate (SDDNa, 0.5 M in DMF) twice and the N-terminal Fmoc group was removed by treatment with 20% piperidine. The resin was washed with DMF, DCM, and DMF (3 times each) and incubated with 1 M HOBt for 5 min. The peptide was cyclized on the resin by treatment with PyBOP/HOBt/DIPEA (5, 5, and 10 equiv, respectively) for 2 h (twice). The resin was then washed with DMF and DCM (3 times each). The cyclic peptides were released from the resin and deprotected by treating the resin with 92.5% TFA, 2.5% triisopropylsilane (TIPS), 2.5% H₂O and 2.5% dimethoxybenzene (DMB) for 3 h. After evaporation of the solvents, the crude peptides were triturated with cold diethylether 3 times and purified by reversed-phase HPLC on a semi-preparative C18 column. For labeling with naphthofluorescein, the purified peptide (˜1 mg) was dissolved in 25 μL of DMF and the pH was adjusted to ˜8 with the addition of 1 M NaHCO₃, and 1 mg of naphthofluorescein 0-succinimidyl ester (NF-OSu) in 25 μL of DMF was added. The mixture was incubated for 2 h at RT. The labeled peptide was purified again by reversed-phase HPLC on a C18 column.

Synthesis of CPP1-3, CPP1-4, CPP1-5, CPP1-8, CPP1-10, CPP1-11, CPP1-12, CPP1-13, CPP1-14, CPP1-15, CPP-16, CPP1-17, CPP1-18, CPP1-19. Peptides were manually synthesized as described above but with the following modifications. After the coupling of Fmoc-L-Dap(methyltrityl[“Mtt” ])—OH, Fmoc-L-Lys(Mtt)-OH, or Fmoc-L-Orn(Mtt)-OH, the Mtt group was removed by treatment with 2% TFA and 2% triisopropylsilane in DCM (6×5 min). The exposed side-chain amino group was treated with the proper carboxylic acid/HATU/DIPEA (4, 4, 8 equiv) in DMF (2 h at RT). The resin was washed with DCM and DMF (3 times each). The allyl group on the α-carboxyl group of the C-terminal L-Glu was then removed by treatment with tetrakis(triphenylphosphine)palladium/phenylsilane (0.3 and 10 equiv, respectively) in DCM for 15 min (3 times). The peptide was then cyclized, deprotected, labeled, and purified as described above.

Synthesis of CPP1-7, CPP1-15. These two peptides were manually synthesized as described above but with the following modifications. Fmoc-L-Dap(Mtt)-OH or Fmoc-L-Lys(Mtt)-OH was used for the coupling of residue “X”. After the cyclization of the peptide, the Mtt group was removed by treatment with 2% TFA and 2% triisopropylsilane in DCM (6×5 min). The exposed side-chain amino group was treated with Fmoc-OSu (for CPP1-7) or decanoic acid/HATU/DIPEA (4, 4, 8 equiv; for CPP1-15) in DMF (2 h at RT).

Synthesis of CPP1-20, CPP1-22, CPP1-23, CPP1-24, and CPP1-25. The peptides were manually synthesized as described above except for the following modifications. Fmoc-L-Asp-O-2-PhiPr or Fmoc-L-Glu-O-2-PhiPr (where 2-PhiPr is 2-phenylisopropyl) was used for the incorporation of residue “X”. After that, the 2-PhiPr group was removed by treatment with 2% TFA and 2% triisopropylsilane in DCM (6×5 min). The exposed side-chain carboxyl group was then amidated by treatment with decylamine/PyBOP/HOBt/DIPEA (1, 5, 5, and 10 equiv) twice in DMF solution. The peptide was cyclized, deprotected, labeled, and purified as described above.

To generate fluorescently labelled peptides, an N^{e}-4-methoxytrityl-L-lysine was added to the C-terminus prior to peptide synthesis. After the solid-phase synthesis was complete but before cleavage, the lysine side chain was selectively deprotected using 1% (v/v) TFA in DCM. The resin was incubated with 5 equiv. of a reactive fluorescent labelling reagent (fluorescein isothiocyanate, Lissamine rhodamine B sulfonyl chloride, or naphthofluorescein succinimidyl ester) and 5 equiv. of DIPEA in DMF overnight. The labeled peptide was deprotected, triturated, purified, and analyzed by MALDI-TOF MS as described above.

### Example 2. Cellular Uptake Efficiency

All of the peptides were labeled with naphthofluorescein (NF) at the Gln side chain through a long, flexible linker, miniPEG-Lys. HeLa cells were treated with 5 μM peptide for 2 h in the presence of 10% FBS and the cytosolic entry of the peptides was quantitated by flow cytometry analysis.

To determine cellular uptake efficiency of the CPPs disclosed herein, HeLa cells were cultured in 12-well plates (1.5×10⁵ cells per well) overnight. The cells were incubated for 2 h with 5 mM naphthofluorescein (NF)-labelled peptide in cellular media. At the end of incubation, the cells were washed with DPBS twice, detached from the plate with 0.25% trypsin, diluted into clear DMEM, pelleted at 250 g for 5 min, washed twice with DPBS, resuspended in DPBS, and analyzed on a BD FACS LSR II flow cytometer. For NF-labelled peptides, a 633-nm laser was used for excitation and the fluorescence emission was analyzed in the APC channel. Absolute cellular uptake efficiency was determined by comparing the concentration (via fluorescence intensity) of the CPP in the cytosol to the concentration in the extracellular medium. Relative cellular uptake efficiency was determined by comparing the cytosolic concentration of the CPP to that cytosolic concentration of the control CPP cyclo(FΦRRRRQ).

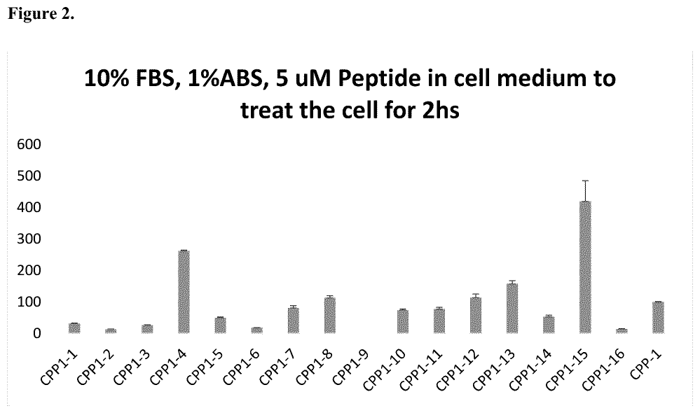

It is clear that the CPP activity increases with the size of the hydrophobic group (FIG. 2). CPP1-4, which contains a decanoyl group is substantially more active than CPP1-2 and CPP1-3, which have hexanoyl and octanoyl groups, respectively. Likewise, the CPP activity also generally increased with the size of the aromatic groups for CPP1-1 and CPP1-5 to CPP1-8. Remarkably, the most active CPP of this series, CPP1-4, showed 2.5-fold higher activity than CPP1, the first cyclic CPP discovered.

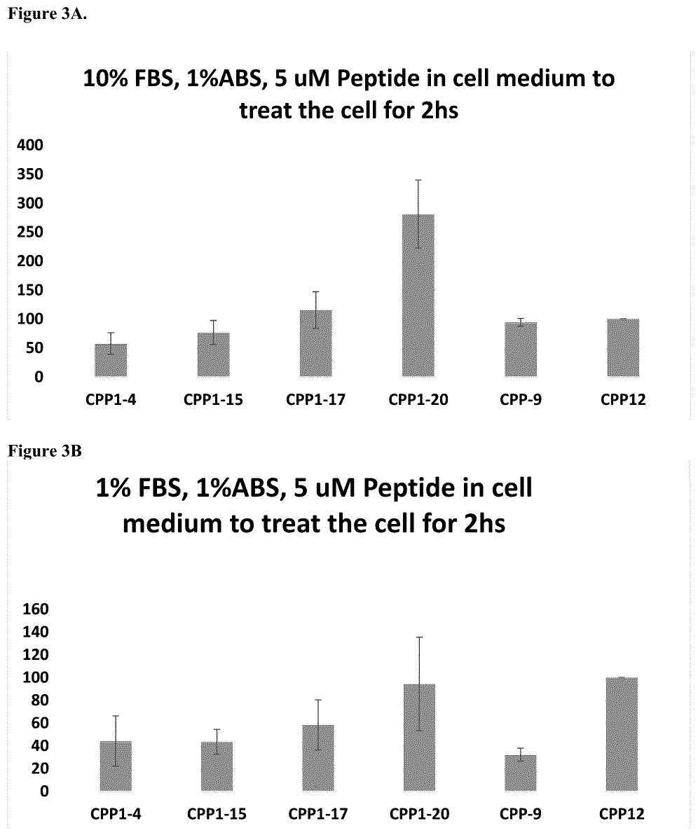

Stereochemical variation of CPP1-4 substantially decreased the CPP activity (FIGS. 2 and 3, CPP1-10 and CPP1-11). Replacement of an arginine with alanine also reduced the CPP activity (CPP1-12 and CPP1-13). Likewise, expansion (CPP1-14) or contraction of the ring size (CPP1-16) greatly decreased the CPP activity. Elongation of the hydrophobic side chain by replacing the Dap residue with lysine or ornithine slightly increased the CPP activity (CPP1-15 and CPP1-17, respectively) (FIGS. 2 and 3). Again, stereochemical variation of CPP1-15 reduced the CPP activity (CPP1-18 and CPP1-19).

The Dap residue of CPP1-4 was replaced with an aspartic acid and the acid functionality was then amidated with decylamine to give CPP1-20 (FIG. 3A). Attempt to synthesize CPP1-21, which contains a glutamic acid in place of the aspartic acid, was not successful. Gratifyingly, CPP1-20 showed ˜5-fold improvement in activity and is ˜3-fold more active than CPP9 and CPP12 (in the presence of 10% FBS). This increase in CPP activity relative to CPP1-4 is likely due to elongation of the hydrocarbon length by one carbon atom (10 vs 9 atoms). Longer alkyl groups presumably insert more deeply into the lipid bilayer, increasing the membrane binding affinity and therefore the CPP activity. It is anticipated that still longer hydrocarbon chains (fatty acyl or alkylamine groups of ≥11 carbon atoms) may further increase the CPP activity. Variation of the stereochemistry of CPP1-20 reduced its CPP activity (CPP1-22 to CPP1-25).

CPP1-4, CPP1-15, CPP1-17, and CPP1-20 show lower sensitivity to serum concentration than CPP9 or CPP12. The cytosolic entry efficiencies of these seven CPPs were tested again with HeLa cells, but in the presence of reduced serum concentration (1% FBS). Under these conditions, CPP12 is the most active CPP, followed by CPP1-20 (94%), CPP1-17 (58%), CPP1-4 (44%), CPP1-15 (43%), and CPP9 (32%) (FIG. 3B). The mean fluorescence intensity (MFI) values of the treated cells were generally much higher than the corresponding values obtained in the presence of 10% FBS. In general, the cyclic CPPs with one hydrophobic group are less sensitive to the FBS concentration than CPP12, likely due to less protein binding by the former and therefore more available for membrane binding and cellular entry. This reduced sensitivity of the new CPPs to serum proteins should result in greater efficacy during in vivo applications.

TABLE 7
Sequences and Cytosolic Entry Efficiencies of Cyclic CPPs

| | | | | | Cellular Entry | |
|---|---|---|---|---|---|---|
| | | SEQ. | | | Efficiency^{b} | |
| | | ID. | Molecular Mass | | In 10% | In 1% |
| Peptide | Sequence^{a} | NO. | Calcd | Obsd | FBS | FBS |
| CPP1 | cyclo(FΦRRRRQ) | 58 | 1827.86 | 1828.837 | 21 | |
| CPP9 | cyclo(fΦRrRrQ) | 59 | 1827.86 | 1828.778 | 94 ± 7 | 32 ± 6 |
| CPP12 | cyclo(FfΦRrRrQ) | 60 | 1974.93 | 1975.839 | 100 | 100 |
| CPP1-1 | cyclo(F^{tBu}RRRRQ) | 61 | 1686.84 | 1687.868 | 6.7 ± 0.4 | |
| CPP1-2 | cyclo(Dap^{Hexan}RRRRQ) | 62 | 1667.83 | 1668.816 | 2.9 ± 0.2 | |
| CPP1-3 | cyclo(Dap^{Octan}RRRRQ) | 63 | 1696.86 | 1696.889 | 5.7 ± 0.6 | |
| CPP1-4 | cyclo(Dap^{Deca}RRRRQ) | 64 | 1723.89 | 1746.906 | 57 ± 19 | |
| CPP1-5 | cyclo(Dap^{1-Pyren}RRRRQ) | 65 | 1797.82 | 1798.836 | 10 ± 20 | |
| CPP1-6 | cyclo(Dap^{3,3-dipheny}RRRRQ) | 66 | 1706.81 | 1707.834 | 3.9 ± 0.4 | |
| CPP1-7 | cyclo(Dap^{Fmoc}RRRRQ) | 67 | 1791.83 | 1792.861 | 17 ± 1 | |
| CPP1-8 | cyclo(Dap^{1-Pyreneb}RRRRQ) | 68 | 1839.86 | 1840.907 | 24 ± 1 | |
| CPP1-9 | Not synthesized | | | | | |
| CPP1-10 | cyclo(Dap^{Deca}RrRrQ) | 69 | 1723.89 | 1724.923 | 21 ± 9 | 24 |
| CPP1-11 | cyclo(Dap^{Deca}rRrRQ) | 70 | 1723.89 | 1724.895 | 20 ± 6 | 29 |
| CPP1-12 | cyclo(Dap^{Deca}ARRRQ) | 71 | 1638.83 | 1639.834 | 26 ± 4 | 34 |
| CPP1-13 | cyclo(Dap^{Deca}RRRAQ) | 72 | 1638.83 | 1639.824 | 32 ± 2 | 33 |
| CPP1-14 | cyclo(Dap^{Deca}RRRRRQ) | 73 | 1880.00 | 1880.979 | 12 ± 1 | 21 |
| CPP1-15 | cyclo(Lys^{Deca}RRRRQ) | 74 | 1765.94 | 1767.638 | 76 ± 21 | 44 ± 11 |
| CPP1-16 | cyclo(Dap^{Deca}RRRQ) | 75 | 1567.79 | 1568.692 | 3.0 ± 0.1 | 25 |
| CPP1-17 | cyclo(Orn^{Deca}RRRRQ) | 76 | 1751.93 | 1752.832 | 115 ± 32 | 58 ± 22 |
| CPP1-18 | cyclo(Lys^{Deca}RrRrQ) | 77 | 1765.94 | 1766.908 | 31 | |
| CPP1-19 | cyclo(Lys^{Deca}rRrRQ) | 78 | 1765.94 | 1766.880 | 12 | |
| CPP1-20 | cyclo(Asp^{Decy}RRRRQ) | 79 | 1737.91 | 1738.830 | 281 ± 59 | 94 ± 41 |
| CPP1-21 | Synthesis failed | | | | | |
| CPP1-22 | cyclo(Asp^{Decy}RrRrQ) | 80 | 1737.91 | 1738.844 | 11 | |
| CPP1-23 | cyclo(Glu^{Decy}RrRrQ) | 81 | 1751.93 | 1752.833 | 1.4 | |
| CPP1-24 | cyclo(Asp^{Decy}rRrRQ) | 82 | 1737.91 | 1738.854 | 8.6 | |
| CPP1-25 | cyclo(Glu^{Decy}rRrRQ) | 83 | 1751.93 | 1752.837 | 34 | |

^{a}Single-letter codes for amino acids. Φ, 2-naphthylalanine. tBu = tert-butanoyl, hexan = hexanoyl, octan = octanoyl, deca = decanoyl, 1-pyren = pyrenol, 3,3-diphenyl = 3,3-diphenoyl, Fmoc = fluorenylmethyloxycarbonoyl, 1-pyreneb = 1-pyrenylbutanoyl, decy = decynoyl
^{b}All values are relative to that of CPP12 (100%).

### Example 4. Cytotoxicity

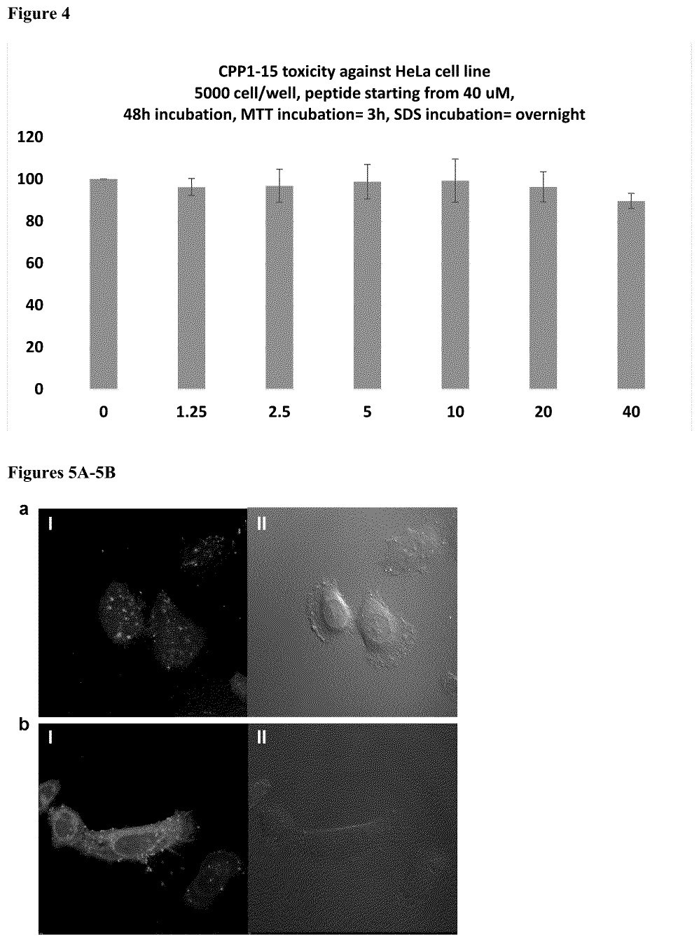

CPP1-15 (no label) was used as a representative and tested against HeLa cells for potential cytotoxicity by the MTT assay. It did not show significant cytotoxicity up to 40 μM concentration (FIG. 4).

### Example 5. Confirmation of Cellular Entry by Confocal Microscopy

CPP1-15 and CPP1-20 were labeled with FITC via a miniPEG-Lys linker. HeLa cells were treated with 5 μM peptide for 2 h, washed, and imaged by live-cell confocal imaging. Both peptides entered HeLa cells efficiently and produced a combination of diffuse and punctate fluorescence, suggesting that they at least partially enter cells by endocytosis followed by endosomal escape (FIGS. 5A-5B). Flow cytometry analysis at different concentrations suggest that at ≥2 μM concentration, these CPPs start to enter cells by direct translocation. Additional experiments are ongoing to further examine their mechanism of cell entry.

The ability of CPP1-20 to directly translocate across the plasma membrane suggests that it may be able to deliver macromolecular cargoes such as proteins across the plasma membrane. Such transporters may have important in vitro and ex vivo applications. Ongoing studies include conjugation of CPP1-20 to EGFP and testing for entry into HeLa cells via direct translocation.

A number of embodiments of the invention have been described. Nevertheless, it will be understood that various modifications may be made without departing from the spirit and scope of the invention. Accordingly, other embodiments are within the scope of the following claims.

1-92. (canceled)

93. A complex comprising a cargo moiety and cyclic peptide according to one of Formula I-A to I-F, wherein at least one atom of the cyclic peptide or at least one lone pair of the cyclic peptide forms a bond to the cargo moiety, the cyclic peptide comprising: 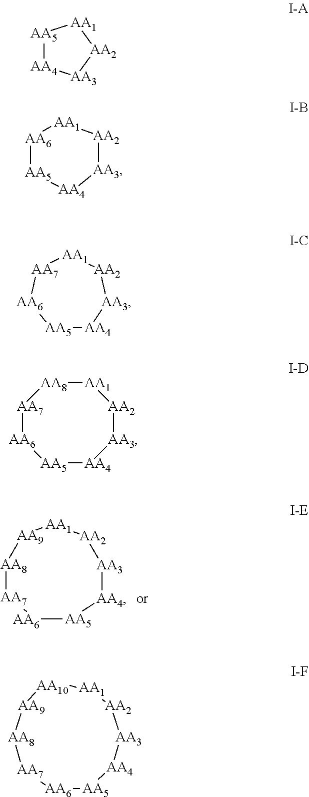
wherein:
each of AA₁, AA₂, AA₃, AA₄, AA₅, AA₆, AA₇, AA₈, AA₉, and AA₁₀, when present, are independently selected from an amino acid; and
wherein:
at least two of the amino acids are arginine;
at least one of the amino acids is an amino acid having a non-aromatic hydrophobic side chain.

94. The complex of claim 93, wherein the cyclic peptide comprises at least three adjacent amino acids having the same or alternating chirality.

95. The complex of claim 93, wherein the amino acids have the same chirality.

96. The complex of claim 93, wherein at least three arginines are present.

97. The complex of claim 93, wherein at least four arginines are present.

98. The complex of claim 93, wherein at least two arginines in the cyclic peptide are adjacent to each other.

99. The complex of claim 93, wherein at least three arginines in the cyclic peptide are adjacent to each other.

100. The complex of claim 93, wherein the at least one amino acid having a non-aromatic hydrophobic side chain is a residue of cysteine, 2,3-diaminopropionic acid, ornithine, lysine, aspartic acid, or glutamic acid; and wherein the residue of cysteine, 2,3-diaminopropionic acid, ornithine, lysine, aspartic acid, or glutamic acid is substituted with a hydrophobic moiety selected from alkyl, alkenyl, alkynyl, acyl, alkylcarboxamidyl, alkoxycarbonyl, carbocyclyl, or heterocyclyl, each of which is optionally substituted with a non-aromatic substituent.

101. A cyclic peptide comprising a structure according to any of Formula III-A to III-L: 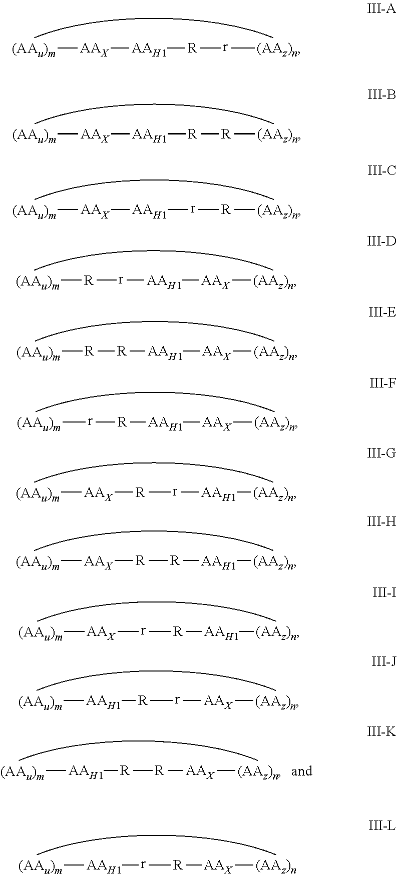
wherein:
AA_{H1} is an amino acid having a non-aromatic hydrophobic side chain;
at each instance AA_{U}, AA_{X}, and AAz are independently any amino acid; and
each of m and n are independently a number from 0 to 6, provided that at least one of m or n is not 0 and the total number of amino acids is from 5 to 10.

102. The cyclic peptide of claim 101, wherein one AA_{U} and one AA_{Z} are arginine.

103. The cyclic peptide of claim 101, wherein AAₓ is selected from a residue of cysteine, glutamine, 2,3-diaminopropionic acid, ornithine, lysine, serine, aspartic acid, glutamic acid, or tryptophan.

104. The cyclic peptide of claim 103, wherein AAₓ is a residue of glutamine.

105. The cyclic peptide of claim 93, wherein the cyclic peptide comprises the sequence:
cyclo(F^{tBu}RRRRQ);
cyclo(Dap^{Hexan}RRRRQ);
cyclo(Dap^{Octan}RRRRQ);
cyclo(Dap^{Deca}RRRRQ);
cyclo(Dap^{1-Pyren}RRRRQ);
cyclo(Dap^{3,3-dipheyl}RRRRQ);
cyclo(Dap^{Fmoc}RRRRQ);
cyclo(Dap^{1-Pyrenb}RRRRQ);
cyclo(Dap^{Deca}RrRrQ)
cyclo(^{DapDeca}rRrRQ);
cyclo(Dap^{Deca}ARRRQ);
cyclo(Dap^{Deca}RRRAQ);
cyclo(Dap^{Deca}RRRRRQ);
cyclo(Lys^{Deca}RRRRQ);
cyclo(Dap^{Deca}RRRQ);
cyclo(Orn^{Deca}RRRRQ);
cyclo(Lys^{Deca}RrRrQ);
cyclo(Lys^{Deca}RrRQ);
cyclo(Asp^{Decy}RRRRQ);
cyclo(Asp^{Decy}RrRrQ);
cyclo(Glu^{Decy}RrRrQ);
cyclo(Asp^{Decyr}RrRQ); or
cyclo(Glu^{Decy}rRrRQ).

106. The complex of claim 93, wherein the cargo comprises one or more detectable moieties, one or more therapeutic moieties, one or more targeting moieties, or any combination thereof.

107. The complex of claim 106, wherein the cargo moiety comprises one or more therapeutic moieties.

108. A pharmaceutical composition comprising a complex of claim 93 in combination with a pharmaceutically acceptable carrier.

109. The pharmaceutical composition of claim 108 formulated for parenteral administration.

110. A method for treating a disease or pathology in a subject in need thereof comprising administering to the subject an effective amount of the pharmaceutical composition of claim 108.

111. The method of claim 110, wherein the disease or pathology comprises cancer, a metabolic disorder or an immune disorder.
